(19) United States
(12) Patent Application Publication
(10) Pub. No.: US 2022/0180967 A1
(43) Pub. Date: Jun. 9, 2022

(54) METHODS AND SYSTEMS FOR GENETIC ANALYSIS

(71) Applicant: Personal Genome Diagnostics Inc., Baltimore, MD (US)

(72) Inventors: John F. Thompson, Baltimore, MD (US); Brett Whitty, Baltimore, MD (US)

(21) Appl. No.: 17/604,958

(22) PCT Filed: Apr. 21, 2020
(86) PCT No.: PCT/US2020/029113

§ 371 Date: Oct. 19, 2021

Related U.S. Application Data
(60) Provisional application No. 62/837,034, filed on Apr. 22, 2019.

(51) Int. Cl. G16B 20/20; G16B 20/40

(52) U.S. Cl. CPC G16B 20/20; G16B 20/40

(57) ABSTRACT

The present disclosure provides computational methods for genetic analysis as well as systems for implementing such analyses. The present disclosure provides methods of genetic analysis which utilize microhaplotypes that are associated with SNPs that are single base pair substitutions (SBSs) in preference to insertion or deletion SNPs. Analysis of such microhaplotypes is useful in forensic genetic applications, sample contamination analysis, and disease analysis, among other applications.

### CROSS-REFERENCE TO RELATED APPLICATIONS

This application claims benefit of priority under 35 U.S.C. § 119(e) of U.S. Ser. No. 62/837,034, filed Apr. 22, 2019, the entire contents of which is incorporated herein by reference in its entirety.

### BACKGROUND OF THE INVENTION

### Field of the Invention

The invention relates generally to genetic analysis and more specifically to methods and systems for analyses of microhaplotypes to determine genetic identity in complex DNA mixtures.

### Background Information

Sequence variation in the human genome is a cornerstone in human identification and forensic applications. Genetic fingerprinting is a forensic technique used to identify individuals by characteristics of their genetic information (e.g., RNA, DNA). A genetic fingerprint is a small set of one or more nucleic acid variations that is likely to be different in all unrelated individuals, thereby being as unique to individuals as are fingerprints.

Sequence variation is useful in genetic analysis for a host of applications such as detection of contamination in a biological sample, forensic analysis, disease detection and population genetics to name a few. Single nucleotide polymorphisms (SNPs) have long been used in genetic analysis for such applications.

DNA contamination in biological samples is a wide spread problem. Contamination can occur at almost every stage of sample collection/processing. For example, slides can be contaminated while cutting, liquids can be inadvertently transferred between tubes, libraries can be mixed, and sample barcodes can be impure or have low quality sequences. Contamination is more likely to be noticeable with samples with low yield and/or poor quality DNA.

SNPCheck™ is a tool for performing batch checks for the presence of SNPs and can be utilized to confirm the presence of DNA contamination in a sample. With “well-behaved” DNA like normal tissue or cfDNA, SNPCheck™ can provide reasonable results because Minor Allele frequencies (MAFs) are nearly all around 0 or 0.5. However, extremely high contamination levels are missed because the MAFs are so high and can approach 0.5. Tumor DNA is not “well-behaved” because extreme copy number variation can lead to MAFs ranging from 0.02 to 0.98. This means that MAFs for contamination and real variants can significantly overlap.

A detection method that is independent or nearly independent of MAF is needed to be able to both detect DNA contamination and further quantitate the amount of contamination in an accurate way.

### SUMMARY OF THE INVENTION

The present disclosure provides methods of genetic analysis which utilize microhaplotypes that are associated with SNPs that are single base pair substitutions (SBSs) in preference to insertion or deletion SNPs. Analysis of such microhaplotypes is useful in forensic genetic applications, sample contamination analysis, and disease analysis, among other applications.

In one embodiment, the disclosure provides a method for genetic analysis which includes: a) identifying SNP sets having at least 3 microhaplotypes in a sample; and b) quantitating the frequency of haplotypes within the SNP sets with more than 2 microhaplotypes.

In another embodiment, the disclosure provides a method for genetic analysis which includes: a) identifying SNP sets having at least 3 microhaplotypes in a sample; and b) quantitating the frequency of the haplotypes within SNP sets with more than 2 microhaplotypes to determine the presence or absence of DNA contamination in the sample.

In yet another embodiment, the disclosure provides a method for genetic analysis which includes: a) identifying SNP sets having at least 3 microhaplotypes in a sample; and b) quantitating the frequency of the haplotypes within SNP sets with more than 2 microhaplotypes to determine the presence or absence of a genetic marker indicative of the disease or disorder.

In still another embodiment, the disclosure provides a method of identifying microhaplotypes in a genome. The method includes: a) identifying a region of interest of the genome; b) detecting SBSs within the region of interest thereby generating multiple sequence variant sets; c) analyzing each variant set for linkage disequilibrium to identify candidate microhaplotypes; and d) identifying candidate microhaplotypes.

In another embodiment, the disclosure provides a method for detecting SNP sets having at least three microhaplotypes from multiple subjects present in a sample. The method includes: a) identifying microhaplotypes in a genome in the sample; b) determining the number of SNP sets having at least 3 microhaplotypes in the sample; and c) quantitating the frequency of the haplotypes within SNP sets with greater than 2 microhaplotypes to determine the presence of DNA from multiple subjects in the sample, thereby detecting DNA from multiple subjects in the sample. In one embodiment, identifying includes: i) identifying a region of interest of the genome; ii) detecting SBSs within the region of interest thereby generating multiple sequence variant sets; and iii) analyzing each variant set for LD to identify microhaplotypes.

In an embodiment, the disclosure provides a method for detecting SNP sets having at least two microhaplotypes from multiple subjects present in a sample. The method includes: a) determining the presence or absence of SNP sets having more than two microhaplotypes in the sample, wherein the SNP sets comprise multiple single base pair substitutions and correspond to a genomic region set forth in Tables 5, 6 and 7; and b) quantitating the frequency of haplotypes within the SNP sets to determine the presence of DNA from multiple subjects in the sample, thereby detecting SNP sets having more than 2 microhaplotypes from multiple subjects in the sample.

In one embodiment the disclosure provides an oligonucleotide panel. The panel includes oligonucleotides for amplifying or hybrid capturing a region of a genome corresponding to one or more genomic regions set forth in Tables 5, 6 and 7.

In another embodiment, the disclosure provides a method of genetic analysis that includes: a) amplifying a region of a genome present in a sample, the region corresponding to a genomic region set forth in Tables 5, 6, and 7 thereby generating an amplicon; and b) sequencing the amplicon to determine the nucleic acid sequence of the amplicon.

In a further embodiment, the disclosure provides a method for detecting a disease or disorder in a subject. The method includes: a) obtaining a sample from the subject; b) identifying microhaplotypes in DNA molecules present in a sample; c) determining the presence or absence of SNP sets having more than 2 microhaplotypes in the sample; and d) quantitating the frequency of haplotypes within SNP sets to determine the presence or absence of a genetic marker indicative of the disease or disorder, thereby detecting the disease or disorder. In one embodiment, identifying includes: i) identifying a region of interest, wherein the region of interest is associated with the disease or disorder; ii) detecting SBSs within the region of interest region of interest thereby generating multiple sequence variant sets; and iii) analyzing each variant set for LD to identify microhaplotypes.

In an embodiment the disclosure provides a genetic analysis system. The system includes: a) at least one processor operatively connected to a memory; b) a receiver component configured to receive DNA analysis information including microhaplotype sequence information generated from PCR amplification of DNA in a DNA sample; and c) an analysis component, executed by the at least one processor, configured to: i) identify microhaplotypes in the sample based on the presence of single base pair substitutions; ii) confirm presence of the number of SNP sets for microhaplotypes in the DNA sample; and iii) quantitate the frequency of genotypes within SNP sets with more than 2 microhaplotypes in the DNA sample.

In a related embodiment the disclosure provides a genetic analysis system configured to perform a method of the disclosure. The system includes: a) at least one processor operatively connected to a memory; b) a receiver component configured to receive DNA analysis information including microhaplotype sequence information generated from PCR amplification of DNA in a DNA sample; and c) an analysis component, executed by the at least one processor, configured to perform a method of the disclosure.

In still another embodiment, the invention provides a non-transitory computer readable storage medium encoded with a computer program. The program includes instructions that, when executed by one or more processors, cause the one or more processors to perform operations that implement a method of the disclosure.

In yet another embodiment, the invention provides a computing system. The system includes a memory, and one or more processors coupled to the memory, with the one or more processors being configured to perform operations that implement a method of the disclosure.

### BRIEF DESCRIPTION OF THE FIGURES

FIG. 1 is a graph showing data generated using the method of the disclosure in one embodiment of the invention.

FIG. 2 is a graph showing data generated using the method of the disclosure in one embodiment of the invention.

FIG. 3 is an image depicting microhaplotype frequency in the presence of contamination in embodiments of the invention.

### DETAILED DESCRIPTION OF THE INVENTION

The present invention is based on innovative methods and systems for genetic analysis of microhaplotypes. Before the present compositions and methods are described, it is to be understood that this invention is not limited to particular methods and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present disclosure provides innovative methods and systems for genetic analysis utilizing microhaplotypes. The methods utilize SBS SNPs and in embodiments SBS changes in low error genomic regions. This allows for increased accuracy in detection of DNA contamination, detection of disease as well as forensic analysis. The methods disclosed herein use SBSs in preference to STRs or insertion/deletion SNPs because the latter have an unacceptably high error rate that affects detection of low levels of contamination in a sample. All of the methods of the disclosure focus on SNP variants with a short genetic distance between them so they can ideally be on a single sequence read. Long read technologies allow longer distances as long as the SNP variants are on a single read. While longer distances can be used, using a paired read leads to a higher error rate and coverage is lower the further away the variants are. Further, certain methods of the disclosure advantageously utilize a two-phase analysis, first to detect contamination and then to quantitate it. Detection of DNA contamination via the method disclosed herein relies on the number of microhaplotypes for each SNP set and/or the frequency of 3^{rd}/4^{th} haplotypes, not on the MAFs of individual SNPs.

Previous investigations have illustrated the utility of multiple closely linked SNP-based markers in anthropology for population relationship and their capacity to provide a plausible explanation for the pattern of recent human variation. In addition, multi-allelic SNPs have been promoted as suitable markers for addressing relevant forensic questions such as family/clan, lineage inference, and individual identification. Aiming to complement current DNA typing tools for forensics and population genetics, the Kidd laboratory proposed a novel type of genetic marker named microhaplotypes (e.g., “microhaps” or MHs). These are short segments of DNA (<300 nucleotides, thus “micro”), characterized by the presence of two or more closely linked SNPs that present three or more allelic combinations (i.e., “haplotypes”) within a population. The short distance between SNPs implies an extremely low recombination rate among them. The level of heterozygosity of the microhaplotypes is dependent upon different factors, including historical accumulation of allelic variants at different positions within the targeted region, incidence of rare crossover events, occurrence of random genetic drift, and/or selection. Since microhaplotypes are multi-SNP haplotypes, they can provide, on a per locus basis, a larger assembly of information than a stand-alone SNP marker.

Further, when variants are near each other on the genome, they tend to be correlated. Each different set of SNPs on a single chromosomal allele is called a haplotype (a set of linked SNP alleles that tend to always occur together (i.e., that are associated statistically)). Because each individual has 2 copies of his/her genome, each person has 2 haplotypes in autosomal chromosomal regions. These haplotypes can be different (heterozygous) or identical (homozygous). As discussed above, a microhaplotype is a short haplotype that is about 300 nucleotides or less or longer distances for long reads. For the purposes of the methods described herein, a microhaplotype is short enough in length such that the variants are on the same sequencing read so can be unambiguously phased. Most microhaplotypes are not particularly useful in genetic analysis since 2 and only 2 microhaplotypes are ever found in a population. However, the methods of the present invention allow for identification of microhaplotypes that can provide statistically useful information such as those microhaplotypes where there can be 3, 4, 5, or even more different haplotypes found among different individuals (but never more than 2 in one individual).

As used herein, a “SNP” is a single-nucleotide substitution of one base (e.g., cytosine, thymine, uracil, adenine, or guanine) for another at a specific position, or locus, in a genome, where the substitution is present in a population to an appreciable extent (e.g., more than 1% of the population).

In certain embodiments, the methods of the disclosure relate to determining and quantitating the presence of DNA contamination in a DNA sample.

In related embodiments, the methods of the disclosure relate to determining whether a sample includes a complex mixtures of DNA from multiple individuals. Such individuals may be mother and offspring, as well as related or unrelated individuals.

Conventional forensics analysis uniquely identifies individual DNA samples through extraction of short tandem repeats (STRs) and/or determination of mitochondrial DNA (mtDNA) sequences. Capillary electrophoresis is often used to quantify STR lengths and mtDNA sequences. This methodology has been proven accurate for individual profile identification.

Of significance to the methods to the disclosure, the ability of these methods to deconvolute complex DNA mixtures into component profiles does not require any prior knowledge of the components. For example, the methods described herein are effective to deconvolute complex DNA mixtures into component profiles without any knowledge of genetic markers or DNA sequences belonging to any individual or component that contributes to any one of the complex DNA mixtures. Thus, one of the superior properties of the methods of the disclosure is that the methods do not require any prior knowledge or data regarding individual profiles, contributors, or components of a complex DNA mixture.

In some aspects, techniques described herein can be used to determine the ethnicity of an individual associated with DNA present in a biological sample.

In embodiments, the disclosure provides a method of identifying microhaplotypes in a genome. The microhaplotypes are useful for use in any of the methods disclosed herein, for example, in detection of sample contamination, disease analysis and/or complex sample deconvolution.

Accordingly, the disclosure provides a method of identifying microhaplotypes in a genome. The method includes: a) identifying a region of interest of the genome; b) detecting SBSs within the region of interest thereby generating multiple sequence variant sets; c) analyzing each variant set for LD to identify candidate microhaplotypes; and d) identifying candidate microhaplotypes.

Also, provided is a method that includes: a) identifying SNP sets having at least 3 microhaplotypes in a sample; and b) quantitating the frequency of haplotypes within the SNP sets with more than 2 microhaplotypes.

Additionally, the disclosure also provides a method that includes: a) identifying SNP sets having at least 3 microhaplotypes in a sample; and b) quantitating the frequency of haplotypes within the SNP sets with more than 2 microhaplotypes to determine the presence or absence of DNA contamination in the sample.

A method for genetic analysis is also provided that includes: a) identifying SNP sets having at least 3 microhaplotypes in a sample; and b) quantitating the frequency of the haplotypes within SNP sets with more than 2 microhaplotypes to determine the presence or absence of a genetic marker indicative of the disease or disorder.

In various embodiments, the methodology of the disclosure may further include quantitating the frequency of SNP sets having at least 3, 4, 5, 6 or more microhaplotypes in the sample. This may be performed to determine the amount of DNA contamination in the sample. In embodiments, as discussed in Example 1, the method further includes calibrating cutoff values for candidate microhaplotypes. Sample contamination can be assessed utilizing determined cutoff values for frequency of candidate microhaplotypes having SNP sets with at least 3, 4, 5, 6, 7, 8 or more microhaplotypes.

The microhaplotypes of the present invention can use different SNP sets but principles of choosing them are the same. As discussed here, the principles include: use of databases such as gnomAD™ (for exons, ˜52% European, 7% East Asian, 6% African), for picking candidate SNPs, 1000 Genomes™ database (˜20% European, 20% East Asian, 26% African) for evaluating LD; selecting a final set of SNPs based on 1000 Genomes frequency (or similar database) of third/fourth haplotypes to equalize variation across ancestries (use of the gnomAD database leads to slightly higher variation among Europeans); variants must be close enough to be on same sequence read; use of single base substitutions, avoiding repeat sequences/indels, to minimize error rate; avoidance of homopolymer and low confidence sequence regions; choice of SNPs in low LD so frequency of 3rd/4th haplotype is high; maximization of distance between SNP sets so information is independent; and test of candidate SNP sets against real samples to ensure high coverage, diverse genotypes, and low rate of 3rd/4th haplotypes in pure samples.

The methodology of the present disclosure may include identification of candidate variant sets for analysis as discussed in Example 1.

This may include identifying a region of interest of the genome and determining the nucleotide sequence of the region for use in analysis. The region of interest is examined for the presence of SBSs. In embodiments, the SBS frequency is typically between about 5-95% which may be determined using a suitable genomic database, for example the gnomAD™ database (gnomad.broadinstitute.org/).

In embodiments, the region of interest utilized optionally includes flanking regions which are also examined for the presence of SBSs with a frequency also determined to be between about 5-95%. In various embodiments, the regions flanking the region of interest include less than about 50, 100, 150, 180 or 200 nucleotide base pairs. In various embodiment, the total length of the region of interest, optionally including flanking regions is less than about 500, 450, 400, 350, 300, 250, 200, 150, 100, 90, 80, 70, 60, 50, 40, 30, 20, 10 base pairs.

In embodiments, the candidate variant pairs that are identified are then examined for LD. This may be performed using the 1000 Genomes™ database (ldlink.nci.nih.gov/?tab=ldhap).

Pairs, triplets, quartets, and the like with at least three haplotypes and the third and greater haplotypes having a total frequency of >1% are then considered as candidates for use. In various embodiments, microhaplotype variant sets were chosen to avoid insertions/deletions because the intrinsic sequencing error rate in such variants is higher and more likely to generate noise. In some embodiments, variants may not be found in the 1000 Genomes™ database and therefore cannot be easily assessed for LD. However, such variants may be utilized if the MAFs observed in the gnomAD™ database suggest it is appropriate.

It will be appreciated that the region of interest may be within a gene, an intron and/or an exon or between genes. Alternatively, the region of interest may be within an exome. In embodiments, the region of interest may include a genetic marker associated with a disease.

In embodiments, the region of interest may include a genetic marker associated with a particular ethnicity.

Utilizing this approach, oligonucleotide panels may be generated for amplifying or hybrid capturing the particular regions which include the microhaplotypes that are identified using the methods of the disclosure. In one embodiment, the oligonucleotide panel includes oligonucleotides for amplifying or hybrid capturing a region of a genome corresponding to one or more genomic regions set forth in Table 5. In another embodiment, the oligonucleotide panel includes oligonucleotides for amplifying or hybrid capturing a region of a genome corresponding to one or more genomic regions set forth in Table 6 or 7.

As such, the disclosure also provides a method of genetic analysis that includes: a) amplifying a region of a genome present in a sample, the region corresponding to a genomic region set forth in Tables 5, 6, and 7, thereby generating an amplicon; and b) sequencing the amplicon to determine the nucleic acid sequence of the amplicon.

As discussed herein, the microhaplotypes identified by the methods of the disclosure may be utilized for various applications, including but not limited to DNA contamination detection, disease analysis, and sample deconvolution (i.e., detection of DNA from multiple subjects or cell types in a single sample).

In one embodiment, the disclosure provides a method for detecting SNP sets having at least three microhaplotypes from multiple subjects present in a sample. The method includes: a) identifying microhaplotypes in a genome of the sample; b) determining the number of SNP sets having at least 3 microhaplotypes in the sample; and c) quantitating the frequency of the SNP sets with greater than 2 microhaplotypes to determine the presence of DNA from multiple subjects in the sample, thereby detecting DNA from multiple subjects in the sample. In one embodiment, identifying includes: i) identifying a region of interest of the genome; ii) detecting SBSs within the region of interest thereby generating multiple sequence variant sets; and iii) analyzing each variant set for LD to identify microhaplotypes.

In another embodiment, the disclosure provides a method for detecting SNP sets having at least three microhaplotypes from multiple subjects present in a sample. The method includes: a) determining the presence or absence of SNP sets having at least three microhaplotypes in the sample, wherein the SNP sets comprise multiple single base pair substitutions and correspond to a genomic region set forth in Tables 5 and 6 and 7; and b) quantitating the frequency of the SNP sets to determine the presence of DNA from multiple subjects in the sample, thereby detecting SNP sets having at least three microhaplotypes from multiple subjects in the sample.

Accordingly, the methods of the disclosure for deconvolution or resolution of a component from a complex DNA mixture may be performed by analyzing a single complex DNA mixture. In certain embodiments of the methods of the disclosure for deconvolution or resolution of a component from a complex DNA mixture, the method may analyze more than one complex DNA mixture. The resolution of DNA profiles using these methods increases as the number of SNP loci increase in the panel used. As used herein, the term complex DNA mixture refers to a DNA mixture comprised of DNA from two, or more contributors. Preferably, the complex DNA mixtures of the methods described herein include DNA from at least 2, 3, 4, 5, 6, 7, 8, 9, 10, 11, 12, 13, 14, 15, 16, 17, 18, 19, 20, or more contributors.

Methods of the disclosure are superior to existing methods of deconvoluting DNA profiles. Notably, applications for the methods described herein are not confined to the context of forensic analysis or DNA contamination detection. For example, the methods of the disclosure may be used for medical diagnosis and/or prognosis. To detect diseases, the region of interest may be chosen such that it includes a genetic marker that is associated with a disease or disease state, such as cancer or a fetal disorder. In this manner, the region of interest may be, for example, on chromosome 21 which allows for diagnosis of trisomy 21, also known as Down syndrome. If a sample is determined to be from a mother and fetus and the 3^{rd} microhaplotype frequency is different on chromosome 21 relative to other chromosomes, this is indicative of a gene copy mutation, e.g., trisomy 21. Other trisomies including chr13 and chr18 trisomy can be detected similarly.

As such, the methods described herein may be used in a variety of ways to predict, diagnose and/or monitor diseases, such as cancer and fetal disorders. Further, the methods may be utilized to distinguish various cell types from one another.

In the field of cancer, biopsy samples often contain many cell types, of which a small proportion may form any part of a tumor. Consequently, DNA obtained from tumor biopsies is another form of complex DNA mixture and may contain somatic variants that arise on a particular DNA molecule. In the case of somatic variation, the limitation to SBSs can be relaxed because the somatic variation could be an indel or other modification that would otherwise be avoided. Moreover, within a tumor, the multitude of cells may be molecularly distinct with respect to the expression of factors indicating or facilitating, for example, vascularization and/or metastasis. A DNA mixture obtained from a tumor sample may also form a complex DNA mixture of the disclosure. In both of these non-limiting examples, the methods of the disclosure may be used to build individual profiles for each cell or cell type that contributes to the complex DNA mixture. Moreover, the methods of the disclosure may be used to deconvolute contributors to a complex DNA mixture. For instance, a complex DNA mixture obtained from a breast cancer tumor biopsy may be used to build an individual profile of the malignant cells. In the same patient, a brain cancer tumor biopsy, this individual profile may be used to deconvolute the contributors to the complex DNA mixture obtained from the brain cancer tumor biopsy to determine, for instance, if a malignant breast cancer cell from that subject metastasized to the brain to form a secondary tumor. This method would resolve a question as to whether the tumors arose independently, or, on the other hand, if these tumors are related.

Accordingly, the disclosure provides a method for detecting a disease or disorder in a subject. The method includes: a) obtaining a sample from the subject; b) identifying microhaplotypes in a DNA molecule present in a sample; c) determining the presence or absence of SNP sets having more than 2 microhaplotypes in the sample; and d) quantitating the frequency of haplotypes within SNP sets to determine the presence or absence of a genetic marker indicative of the disease or disorder, thereby detecting the disease or disorder. In one embodiment, identifying includes: i) identifying a region of interest, wherein the region of interest is associated with the disease or disorder; ii) detecting SBSs within the region of interest region of interest thereby generating multiple sequence variant sets; and iii) analyzing each variant set for LD to identify microhaplotypes.

In various embodiments, a genome is present in a biological sample taken from a subject. The biological sample can be virtually any type of biological sample, particularly a sample that contains DNA. The biological sample can be a germline, stem cell, reprogrammed cell, cultured cell, or tissue sample which contains 1000 to about 10,000,000 cells or a fluid with circulating DNA. In embodiments, the sample includes DNA from a tumor or a liquid biopsy, such as, but not limited to amniotic fluid, aqueous humour, vitreous humour, blood, whole blood, fractionated blood, plasma, serum, breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, prostatic fluid, nipple aspirate fluid, lachrymal fluid, perspiration, cheek swabs, cell lysate, gastrointestinal fluid, biopsy tissue and urine or other biological fluid. In one embodiment, the sample includes DNA from a circulating tumor cell. It is possible to obtain samples that contain numbers of cells, even a single cell, in embodiments that utilize an amplification protocol such as PCR. The sample need not contain any intact cells, so long as it contains sufficient biological material (e.g., DNA) to perform genetic analysis of one or more regions of the genome.

In some embodiments, a biological or tissue sample can be drawn from any tissue that includes cells with DNA or a fluid with circulating DNA. A biological or tissue sample may be obtained by surgery, biopsy, swab, stool, or other collection method. In some embodiments, the sample is derived from blood, plasma, serum, lymph, nerve-cell containing tissue, cerebrospinal fluid, biopsy material, tumor tissue, bone marrow, nervous tissue, skin, hair, tears, urine, fetal material, amniocentesis material, uterine tissue, saliva, feces, or sperm. Methods for isolating PBLs from whole blood are well known in the art.

As disclosed above, the biological sample can be a blood sample. The blood sample can be obtained using methods known in the art, such as finger prick or phlebotomy. Suitably, the blood sample is approximately 0.1 to 20 ml, or alternatively approximately 1 to 15 ml with the volume of blood being approximately 10 ml. Smaller amounts may also be used, as well as circulating free DNA in blood. Microsampling and sampling by needle biopsy, catheter, excretion or production of bodily fluids containing DNA are also potential biological sample sources.

In the present invention, the subject is typically a human but also can be any species, including, but not limited to, a dog, cat, rabbit, cow, bird, rat, horse, pig, or monkey.

The method of the disclosure utilizes nucleic acid sequence information, and can therefore include any method for performing nucleic acid sequencing including nucleic acid amplification, polymerase chain reaction (PCR), nanopore sequencing, 454 sequencing, insertion tagged sequencing. In embodiments, the methodology of the disclosure utilizes systems such as those provided by Illumina, Inc, (including but not limited to HiSeg™ X10, HiSeg™ 1000, HiSeg™ 2000, HiSeg™ 2500, Genome Analyzers™, MiSeg™° NextSeq, NovaSeq systems), Applied Biosystems Life Technologies (SOLiD™ System, Ion PGM™ Sequencer, ion Proton™ Sequencer) or Genapsys or BGI MGI and other systems. Nucleic acid analysis can also be carried out by systems provided by Oxford Nanopore Technologies (GridiON™, MiniON™) or Pacific Biosciences (Pacbio™ RS II or Sequel I or II). Importantly, in embodiments, sequencing may be performed using any of the methods described herein. When a long read technology such as PacBio™ or Oxford Nanopore™ is used, the length restrictions on the DNA are loosened and SNPs can be further apart consistent with the longer read lengths.

The present invention includes systems for performing steps of the disclosed methods and is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions.

Methods for genetic analysis according to various aspects of the present invention may be implemented in any suitable manner, for example using a computer program operating on the computer system. An exemplary genetic analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The genetic analysis system according to various aspects of the present invention and its various elements provide functions and operations to facilitate genetic analysis, such as data gathering, processing, analysis, reporting and/or diagnosis. For example, in the present embodiment, the computer system executes the computer program, which may receive, store, search, analyze, and report information relating to the human genome or region thereof. The computer program may comprise multiple modules performing various functions or operations, such as a processing module for processing raw data and generating supplemental data and an analysis module for analyzing raw data and supplemental data to generate quantitative assessments of contamination or a disease status model and/or diagnosis information.

The procedures performed by the genetic analysis system may comprise any suitable processes to facilitate genetic analysis and/or disease diagnosis. In one embodiment, the genetic analysis system is configured to establish a disease status model and/or determine disease status in a patient. Determining or identifying disease status may comprise generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

The genetic analysis system suitably generates a disease status model and/or provides a diagnosis for a patient based on genetic data and/or additional subject data relating to the subjects. The genetic data may be acquired from any suitable biological samples as well as databases storing genetic information.

The following example is provided to further illustrate the advantages and features of the present invention, but it is not intended to limit the scope of the invention. While this example is typical of those that might be used, other procedures, methodologies, or techniques known to those skilled in the art may alternatively be used.

### EXAMPLES

### Example 1

### Detection of Sample Contamination

In this example, the methodology of the present disclose was utilized to detect sample contamination. The following provides an in-depth discussion of the method and process used for detection.

Identification of Candidate Variant Sets.

For each region of interest, the regions targeted for sequencing along with an additional bordering region (up to 100 bp) was examined for SBS with a frequency of 10-90% according to the gnomAD™ database (gnomad.broadinstitute.org/). Once a variant was found that was not in a low confidence region, the neighboring 180 bp in both directions was examined for additional SBSs with a frequency of 5-95%. These cutoffs may vary depending on the type of sample to be analyzed for various panels and the number of SNP sets required. All such variant pairs were then examined for LD using 1000 genomes data (ldlink.nci.nih.gov/?tab=ldhap). Pairs, triplets, etc., with at least three haplotypes and the third and greater haplotypes having a total frequency of >1% were considered as candidates for use. These cutoffs could be expanded to include additional variant sets if necessary or constricted to retain only the most informative variant sets and minimize noise. For example, variant sets were chosen to avoid insertions/deletions because the intrinsic sequencing error rate in such variants is higher and more likely to generate noise. Similarly, other sequence contexts could be favored based on error rates. Furthermore, some variants were not found in the 1000 Genomes™ database so could not be assessed for LD but were advanced for candidate testing if the MAFs observed in gnomAD™ suggested they might be appropriate. While SNPs could in theory be present as far away as paired read partners, SNPs located closer to each other and covered by single reads were chosen to simplify analysis.

Characterization of Candidate Variant Sets.

The candidate variant sets were further evaluated in real samples to ensure that there were enough reads with both/all variants on the read such that a phased haplotype could be generated. A cutoff of 100× median coverage for each SBS was used so that all or nearly all SNP sets could be included in each comparison. High coverage is necessary to maximize sensitivity of the analysis. For other panels, the exact set of SBSs used will vary depending on the panel to be interrogated. Furthermore, some sequence contexts have higher error rates than others and use of those variants could lead to additional, artifactual microhaplotypes. Variant sets prone to too many third/fourth microhaplotypes in purportedly pure samples were eliminated from use because they could generate a high level of noise relative to signal.

A set of 106 variants was chosen for use with a 507 gene panel (Table 5) based on high coverage and low background noise level. To the extent possible, distance between SBS sets was maximized to minimize redundant information. The MAFs listed for SBSs in this table were obtained from “All Populations” of 1000 Genomes™ database and are different than the original MAFs obtained from gnomAD™

Estimating Contamination Levels.

Because any sample could, in theory, be contaminated, it was necessary to characterize samples prior to use for calibration so that the process could start with pure samples. Furthermore, the variant and microhaplotype frequencies can vary significantly across ethnicities so it is useful to characterize samples with different ethnicities to ensure that a given set of SBSs will work with all samples and contaminants. For this data set, five African, five Asian, and six European (all self-identified) were selected based on coverage of at least 105/106 variant sets and no more than 2 variant sets with >2 microhaplotypes. These samples and their characteristics are shown in Table 1. The European samples have a non-significantly lower number of single microhaplotype SBSs.

TAB LE 1
Samples used for calibration.

| Sample | 1 MH | 2 MH | 3 MH | 4 MH | Total | Ethnicity |
|---|---|---|---|---|---|---|
| AATF094T | 44 | 62 | 0 | 0 | 106 | Afri |
| AATF217T | 57 | 49 | 0 | 0 | 106 | Afri |
| AATF218T | 56 | 49 | 1 | 0 | 106 | Afri |
| AATF219T | 47 | 59 | 0 | 0 | 106 | Afri |
| PGRD00454T | 66 | 39 | 0 | 1 | 106 | Afri |
| Mean | 54 | 51.6 | 0.2 | 0.2 | 106 | |
| AATF355T | 49 | 56 | 1 | 0 | 106 | Asian |
| AATF595T | 57 | 47 | 2 | 0 | 106 | Asian |
| AATF597T | 59 | 47 | 0 | 0 | 106 | Asian |
| AATF731T | 45 | 60 | 0 | 1 | 106 | Asian |
| AATF735T | 58 | 46 | 1 | 1 | 106 | Asian |
| Mean | 53.6 | 51.2 | 0.8 | 0.4 | 106 | |
| AATF110T | 42 | 61 | 1 | 1 | 105 | Euro |
| AATF375T | 48 | 56 | 2 | 0 | 106 | Euro |
| AATF389T | 45 | 60 | 1 | 0 | 106 | Euro |
| AATF391T | 57 | 49 | 0 | 0 | 106 | Euro |
| AATF417T | 47 | 58 | 1 | 0 | 106 | Euro |
| AATF088T | 56 | 49 | 1 | 0 | 106 | Euro |
| Mean | 49.2 | 55.5 | 1 | 0.17 | 105.8 | |

To mimic contamination in silico, unfiltered fastQ™ reads from pure samples were computationally mixed with other samples in order to generate artificially “contaminated” samples. For a targeted contamination of X %, 100-X % of the reads from the principle sample were mixed with X % of the reads from the “contaminant”. These mixed samples were then run through the pipeline and aligned and called using our standard methods. The number of haplotypes at each SBS set and their frequency was counted and tabulated for each sample. The frequency of the third haplotype for each SBS set, if any, was then examined within each sample and the minimum, maximum, median, and mean calculated for each set of 3rd haplotype frequencies. The mixes were then examined to see how well contamination could be predicted by these parameters.

Prior to examining the results in detail, multiple technical and biological confounding factors were considered for how they may affect results. As observed with even the “pure” samples, there is technical noise that leads to a small number of 3rd/4th haplotypes. In order to avoid these interfering with contamination detection, a minimum number of 3rd/4th haplotypes was set. The desired level of contamination detection is at the level of 1-2% so the minimum number of 3rd/4th haplotypes was chosen as being in the 5-10 range. This avoids the issue of having low level technical noise being misassigned as contamination.

TABLE 2
Number of SBS sets with > 2 Microhaplotypes (n = 70 each).

| % Contam | 0.5 | 1 | 2 | 5 | 10 |
|---|---|---|---|---|---|
| Minimum | 2 | 5 | 10 | 13 | 15 |
| Median | 8 | 13 | 19 | 23 | 24 |
| Maximum | 18 | 23 | 31 | 32 | 35 |

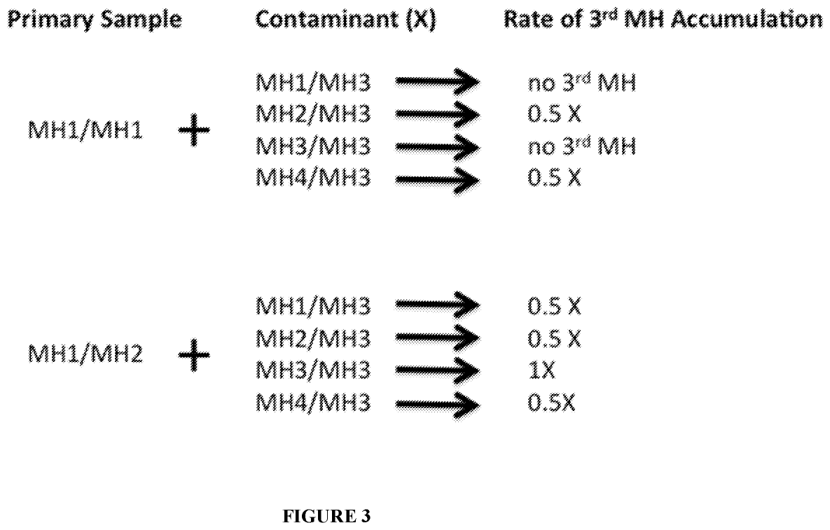

The percent of SNPs with >2 microhaplotypes determines whether a sample is contaminated but it is relatively insensitive to the degree of contamination. Because the %>2 microhaplotype value rapidly achieves a maximum, contamination of 2% vs 5% vs 20% appear very similar when looking only at this parameter. To circumvent this issue, we have used the MAF for the third haplotype for quantitating the level of contamination. This value can be misleading at the low contamination due to technical artifacts. It can appear anomalously high due to the possibility that the contaminating DNA could contribute two copies of the third haplotype, making contamination appear to be 2× higher than reality (FIG. 3). Extreme copy number variation often present in tumor samples can also affect apparent contamination in either direction, depending on which haplotype is in excess. This is not typically a problem with normal DNA but can be severe with tumor DNA. To avoid these issues, we use the median MAF for the third haplotype to minimize the contributions of either abnormally high or low MAFs. There is additional information found in the allele frequencies for the 2nd and 4th microhaplotype though this data was not used for the calculation. More complex analyses of haplotype frequencies can be used if there are enough sets that can be examined.

For samples having above a set number of 3rd/4th haplotypes, a variety of factors could interfere with accurate frequency determination. In the calibration series, one technical issue is whether the nominal contamination level is actually accurate. Though the number of reads added can be precisely controlled, each sample has different properties in terms of DNA quality that may affect the functional level of contamination. Samples with divergent DNA lengths due to different DNA qualities or different fractions of on-target reads due to different capture efficiencies will have different functional levels of contamination because the frequency of SNP sets appearing on the same read is dependent on the length. This would mean that 1% added reads may be functionally equivalent to 0.5% or 2% or anywhere in between. For this reason, each sample and its contaminant were interchanged as sample and contaminant in parallel. Thus, this normalizes quality differences to some extent and provides a better estimate of the functional level of contamination. When these methods are applied to real samples, functional rather than stoichiometric contamination is more important when considering the likelihood that incorrect variant calls could be made.

There are also biological reasons for quantitation issues. A pure sample could have one or two microhaplotypes at each SBS set and the incoming contaminants one or two microhaplotypes could match one, two or neither of the primary sample's microhaplotypes. When contamination is low and the signal just emerging, the new 3rd haplotypes would preferentially be composed of double contributions that do not match the sample's microhaplotypes while there will be a mix of single/double contributions at higher contamination levels. Thus, one should not expect a simple, linear relation between level of contamination and the frequencies of various haplotypes. Superimposed on this difficulty is the occurrence of extensive copy number variation among tumor samples that can also have a major impact on haplotype frequency. Because of these caveats, an empirical estimation of contamination was used because low contamination levels will be overestimated and high contamination levels underestimated if one looks simply at the 3rd haplotype frequencies. With many more variant sets at very high coverage levels, it would be possible to fit the frequency data to better estimate functional contamination. As shown in Table 3, ˜2% is the region where the over- and undercounting balance out to yield a relatively accurate contamination estimation with this set of SNPs and coverage conditions. Since this is around the level at which we would like to set sensitivity, median frequency of the 3rd haplotype will be used as an approximation of the level of contamination, realizing that venturing far from 2% could lead to issues with accuracy. For accurate estimation of other contamination levels, it will be necessary to examine more mixes as has been done with other SBS sets.

TABLE 3
Median frequency of 3rd Haplotypes by ethnicity.

| | Freq of 3^{rd} Haplotype | | |
|---|---|---|---|
| % Contamination | Afri | Asian | Euro |
| 0.5 | 1.0 | 1.2 | 1.2 |
| 1 | 1.2 | 1.4 | 1.7 |
| 2 | 1.8 | 2.4 | 2.6 |
| 5 | 4.1 | 4.4 | 4.9 |
| 10 | 7.0 | 7.7 | 8.0 |

Applications to real samples.

The samples used in the in silico contaminant mixes were chosen based on their high quality. Unfortunately, there is much greater variation in real samples so it is necessary to set criteria for which samples can be analyzed and how that analysis should be done. Ideally, all samples would have >100× coverage at all 106 SBS sets but this is often not the case. Missing SBS sets leads to inconsistent comparisons and low coverage at particular SBSs may lead to grossly overestimated or missing 3rd haplotype frequencies. Thus, 1000 samples were run through the standard pipeline to examine microhaplotype data. Of these 1000 samples, 151 samples had failed standard quality control metrics, leaving 849 for microhaplotype analysis. In order for an SBS to be counted, we require a minimum coverage of 20. The vast majority of samples (709) have data for all 106 SBS sets. However, there are samples with significantly fewer SBS sets meeting the minimum criteria. The point at which more samples fail than pass other quality control metrics is 100 SBS calls. Thus, for the analyses below, only the 825 passing samples with >100 SBS calls are used. Of these 825 samples, 24 failed the previously used SNPCheck™ method for monitoring sample contamination.

Table 4 shows the effects of varying the cutoffs on contamination detection for these 825 samples. Samples pass by either having fewer than the cutoff number of >2 microhaplotype SBS sets or having a 3rd microhaplotype median MAF below a set threshold. Based on the in silico experiments above, that number of SBS sets with >2 microhaplotypes should be in the 5-10 range with these microhaplotypes. In addition, even if there are more than the cutoff number of microhaplotypes, samples with a median 3rd haplotype frequency of <1.5% are also deemed to pass. Using these cutoffs, 804-811 samples pass including 18-19 samples that failed SNPCheck™. If the 3rd haplotype frequency is 2-4%, it is optional that the sample be checked to see if that level of contamination would cause a problem based on the observed somatic mutation frequency. 4-5 of these 11-18 samples failed SNPCheck™ Samples with >4% 3rd microhaplotype frequency would fail. In all cases, this would be three samples, 1 of which failed SNPCheck™. In addition to the 825 passing runs described above, SNPCheck™ had been run on samples that failed other QC metrics or had too few SBSs called in the microhaplotype method of the disclosure. Of the 4 QC and SNPCheck™-failed samples, 3 failed the microhaplotype method with contamination >10%. Of the 7 SNPCheck™-failed samples which would not typically be evaluated by the microhaplotype with fewer than 101 SBSs called, 4 also failed by the microhaplotype method regardless of cutoffs while another one would have failed with some cutoff values.

TABLE 4
Comparison of Microhaplotypes to SNPCheck ™.

| | | # | | # | | # | | # | |
|---|---|---|---|---|---|---|---|---|---|
| | Suggested | Samples | Failed | Samples | Failed | Samples | Failed | Samples | Failed |
| Category | Status | (cutoff 5) | SNPCheck ™ | (cutoff 6) | SNPCheck ™ | (cutoff 8) | SNPCheck ™ | (cutoff 10) | SNPCheck ™ |
| <MH | Pass | 652 | 16 | 701 | 16 | 746 | 17 | 779 | 19 |
| Cutoff | | | | | | | | | |
| Median | Pass | 152 | 2 | 107 | 2 | 64 | 1 | 32 | 0 |
| <2% | | | | | | | | | |
| Median | Check | 13 | 2 | 9 | 2 | 7 | 2 | 7 | 2 |
| 2-3% | | | | | | | | | |
| Median | Check | 5 | 3 | 5 | 3 | 5 | 3 | 4 | 2 |
| 3-4% | | | | | | | | | |
| Median | Fail | 1 | 0 | 1 | 0 | 1 | 0 | 1 | 0 |
| 4-5% | | | | | | | | | |
| Median | Fail | 2 | 1 | 2 | 1 | 2 | 1 | 2 | 1 |
| >5% | | | | | | | | | |

A perfect match between the method of the invention and SNPCheck™ was not expected. SNPCheck™ fails some tumor samples with very high copy number variation by calling pure samples contaminated, leading to false positives. False negatives are also known to arise when the level of contamination is very high and that variation is misinterpreted as germline variation.

Contamination Detection in Exomes.

Many of the SBSs used in the 507 gene panel are in non-coding regions so are of no value in an exome analysis. Thus, a new set of SBSs was chosen for examination of exomes. Because exome coverage is lower on a per ROI basis, it is more important to capture variants with as much of the coverage as possible. Thus, SBS sets were chosen with a shorter inter-variant spacing and localized closer to the exons than in the 507 gene panel. Because there are so many more ROIs, efforts were made to include more informative SBSs and chosen in ROIs that had higher than average coverage. These were then examined in a set of exome data and SBSs with >80 median coverage and diverse haplotypes chosen for use in the panel. These SBS sets are listed in Table 6. Using methods similar to those described above, two exomes suspected to be contaminated were examined and found to be >15% contaminated using this SBS set.

With the initial set of microhaplotypes used for the 507-gene panel, differences were observed in sensitivity among different ancestry groups. This issue was likely caused by both the biases in the databases used to select microhaplotype sets but also by the differences in the heterozygosity rate among different ancestries. To correct for this, population haplotype frequencies from the 1000 genomes project were used to balance the 3rd/4th haplotype frequencies so they were approximately equal across all ancestries. The frequency of 3rd/4th haplotypes among SNP sets was summed and SNP sets which contributed to excess frequency in over-represented ancestries were dropped. This allowed the generation of a set of microhaplotypes such that the expected average number of 3rd/4th haplotypes is the same for those with East Asian, African, and European ancestry. It was not possible to simultaneously generate the same frequencies for the other two 1000 genome ancestries, Admixed American and South Asian. Both of these ancestries had higher 3rd/4th microhaplotype frequencies than the other three so contamination should be easily detected using the same thresholds as the other ancestries.

To further improve performance characteristics, efforts were made to choose only microhaplotype sets with high coverage and low noise among pure samples. Minimum mean coverage for SNP sets was raised from 100 to 250. High coverage, however, is a double-edged sword. While it allows greater sensitivity and higher accuracy, it can also generate artifactual 3rd haplotypes caused by inherent sequencing errors that are typically around the level of 0.1%. To minimize the impact of such technical errors, low frequency haplotypes can be eliminated from consideration. The level at which this should be set can be optimized based on the coverage and sequencing quality. For these experiments, the threshold was set at 0.2% where any haplotype with a frequency below 0.2% was not considered as real. Other thresholds can be used depending on the sequence quality and other factors.

In addition, more SNP sets were used to enhance the signal and allow more precision in contamination estimates. Based on these considerations, 164 SNP sets were chosen for a second microhaplotype panel that meets all these criteria. 51 of these SNP sets were also present in the first panel and both sets are listed in Table 7 with locations, dbSNP numbers, and 1000 genome frequencies of 3rd/4th haplotypes.

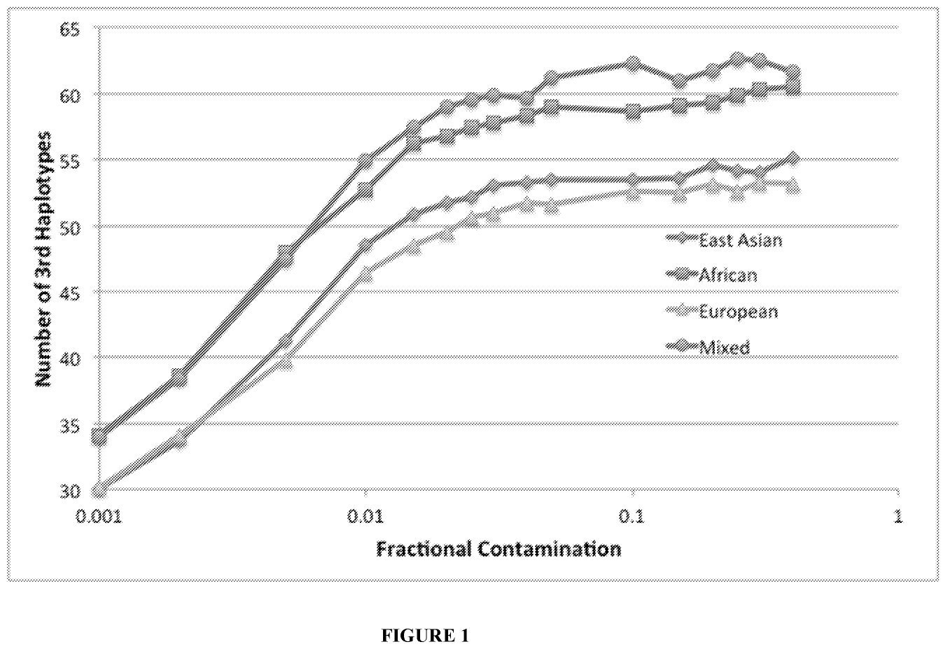

As discussed above, generation of samples with precise levels of contamination is extremely challenging. In silico combination of samples provides a mixed sample with exact levels of contamination but the functional impact is not necessarily precise. Because detection of microhaplotypes is dependent on the length of sequenced molecules, samples with the same fractional component but different DNA quality will have differential impacts on microhaplotype frequencies. To minimize the impact of this, samples were analyzed in pairs, interchanging “sample” and “contaminant” and results then averaged within each pair. 15 such pairs for each category (African, East Asian, European, and Mixed) were then analyzed for the number of 3rd/4th microhaplotypes as a function of contamination level. As shown in FIG. 1, the 3^{rd}/4^{th} MH number for individuals of East Asian and European ancestry were nearly superimposable. The 3^{rd}/4^{th} MH number for individuals of African-American ancestry and mixes of ancestries were higher than East Asian/European but similar to each other. The African-American discrepancy is likely due to the composition of the 1000 genomes African panel which includes 5 sub-groups from Africa and 2 from African-Americans. These two are admixed to some extent and thus generate higher numbers than the other groups. The combination of more even 3rd/4th microhaplotype frequencies and larger number of microhaplotype sets tested will provide more robust identification of contaminated samples.

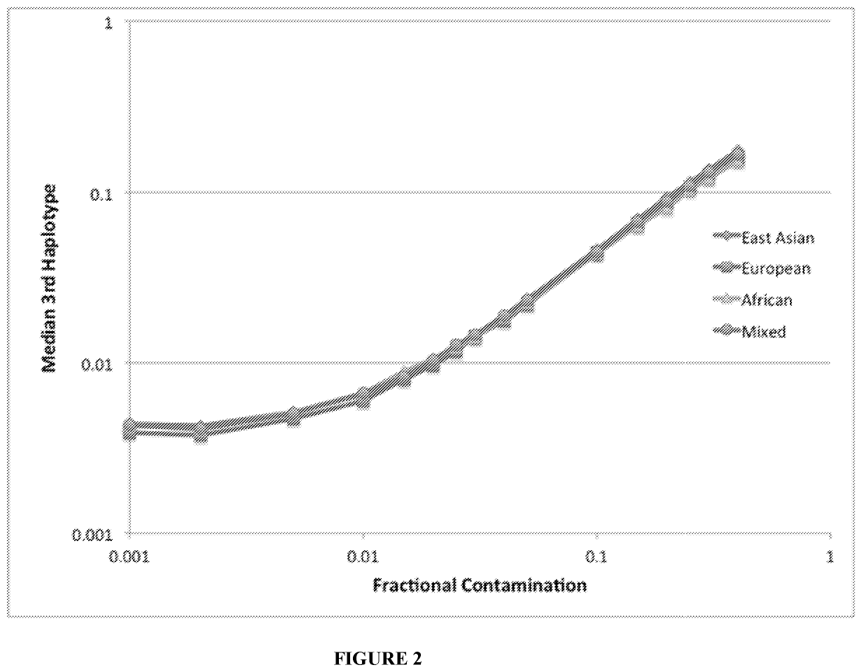

Even though the number of 3rd/4th microhaplotypes varies slightly among different ancestries, the median 3rd microhaplotype frequency as a function of contamination level is nearly identical among those ancestries, including samples mixed from different ancestries (FIG. 2). This relation is linear starting at around 1%. Contamination levels below 1% are impacted heavily by sequencing artifacts as well as the potential presence of additional contaminating DNAs beyond the intended one. Above 1%, the observed median frequency is roughly half the contamination level. This is expected based on the manner in which 3^{rd} MHs are generated, as shown in FIG. 3. At higher levels of contamination this begins to drop off due to a number of factors including the chance that the 3rd microhaplotype may actually be from the sample rather than the contaminant.

Using the relation of contamination level=2×Median 3rd microhaplotype level, the detection of contamination levels at different levels is shown in Table 8 for each ancestry. The patterns are similar with a decreasing fraction of samples being detected at higher contamination levels when the predicted contamination level is twice the 3rd microhaplotype level. This table provides guidance as to where thresholds need to be set to achieve near 100% detection of contamination at a given level. For example, if one wishes to detect nearly all samples contaminated at 2%, setting a cutoff of 3rd microhaplotype=0.75% will detect 97% of samples contaminated at 2% while also including 82% of samples contaminated at 1.5% and only 15% of samples contaminated at 1% and none contaminated at 0.5%. Choice of thresholds can be done based on relative level of false positives and false negatives.

### Example 2

### Using Microhaplotypes for NIPT Detection of Chromosomal Abnormalities

Non-Invasive PreNatal testing (NIPT) for chromosomal abnormality detection is carried out by taking a blood sample from the mother and assessing it for circulating fetal DNA in the presence of a large background fraction of maternal DNA. Typically, sequence reads are simply aligned and the number aligning to each chromosome counted. If there is an excess of reads aligning to chromosomes most susceptible to trisomy (usually chr13, chr18 and chr21), a positive diagnosis is made. This test is typically done at week 10 or later when the amount of fetal DNA in the maternal blood is sufficient for test accuracy. Use of microhaplotypes will allow testing to be done earlier because more accurate quantitation is possible at lower DNA concentrations and provide a more accurate result due to independence from benign copy number variation pre-existing in the mother that can lead to interpretation errors.

The behavior of NIPT samples will be more straightforward than for tumor samples for two reasons. Firstly, the complication of extensive copy number variation will be less of an issue. Secondly, one of the fetal haplotypes will be already present in the mother and the incoming 3rd haplotype from the father will be single copy only so will not be overcounted at low levels. Thus, a more predictable increase in frequency would be expected.

For most trisomy 21 cases, the extra chromosome arises from the mother, deflating the contribution of the new paternal haplotype on that chromosome. Thus, the paternal haplotype frequency on unaffected chromosomes would be determined and compared to the paternal haplotype frequency on potentially affected chromosomes. Because many SBS sets would be available for use, it will be straightforward to generate a list of well-behaved SBSs. These could be enriched via target capture or PCR amplification to allow earlier detection than is currently possible. Unbiased PCR amplification of DNA for typical NIPTs is challenging because slight non-linearities can have an impact on quantitation. Because the microhaplotype method is not simply counting the number of reads but rather looking at the ratio of microhaplotypes, it is less susceptible to amplification biases. Accuracy can be further enhanced by selecting SBS sets that are less prone to sequencing errors or by choosing multi-SBS sets that generate 2 or more sequence changes going from the maternal microhaplotype to the paternal microhaplotype. In addition, the fetal fraction of DNA can be readily determined via examination of the frequencies of genotypes in SNP sets with 3 microhaplotypes. The fetal fraction will be twice the 3^{rd} microhaplotype frequency. Knowledge of the fetal fraction and its variation will provide more accurate determinations of whether a test result is valid or indeterminate.

In order to determine trisomy or other DNA copy-number abnormality, the 3^{rd} microhaplotype frequencies from different regions are compared. If the third microhaplotype frequency from any large genomic region (partial or full chromosome) is different than the frequency of other genomic regions it will signify trisomy or other amplification (increased 3^{rd} microhaplotype frequency) or deletion (no 3^{rd} microhaplotypes).

TABLE 5
SBS sets for the 507 gene panel.

| | | | | | Middle | 3rd | 4th + | SNP1 | SNP2 | SNP3 |
|---|---|---|---|---|---|---|---|---|---|---|
| Location | Length | SNP1 | SNP2 | SNP3 | Pos 1 | MH | MH | MAF | MAF | MAF |
| chr1:120057158- | 89 | rs6203 | rs45609334 | | | 0.167 | | 0.367 | 0.167 | |
| 120057246 | | | | | | | | | | |
| chr1:156846120- | 114 | rs1800880 | rs6334 | | | 0.213 | | 0.232 | 0.213 | |
| 156846233 | | | | | | | | | | |
| chr1:226589833- | 126 | rs1805407 | rs1805404 | | | 0.218 | | 0.263 | 0.218 | |
| 226589958 | | | | | | | | | | |
| chr1:23885498- | 102 | rs11574 | rs2067053 | | | 0.109 | | 0.109 | 0.464 | |
| 23885599 | | | | | | | | | | |
| chr10:104386934- | 86 | rs17114803 | rs12414407 | | | 0.246 | | 0.246 | 0.280 | |
| 104387019 | | | | | | | | | | |
| chr10:43615505- | 129 | rs2472737 | rs1800863 | | | 0.173 | | 0.173 | 0.172 | |
| 43615633 | | | | | | | | | | |
| chr10:70332580- | 93 | rs10823229 | rs12773594 | | | 0.172 | | 0.259 | 0.172 | |
| 70332672 | | | | | | | | | | |
| chr11:534197- | 46 | rs41258054 | rs12628 | | | 0.077 | | 0.077 | 0.297 | |
| 534242 | | | | | | | | | | |
| chr11:8246326- | 18 | rs34544683 | rs3816490 | | | 0.158 | | 0.158 | 0.232 | |
| 8246343 | | | | | | | | | | |
| chr12:121416622- | 29 | rs1169289 | rs1169288 | | | 0.138 | | 0.428 | 0.298 | |
| 121416650 | | | | | | | | | | |
| chr12:121431272- | 29 | rs2071190 | rs1169301 | | | 0.252 | | 0.252 | 0.319 | |
| 121431300 | | | | | | | | | | |
| chr12:121435427- | 49 | rs2464196 | rs2464195 | | | 0.042 | | 0.318 | 0.360 | |
| 121435475 | | | | | | | | | | |
| chr12:121437114- | 108 | rs55834942 | rs1169304 | | | 0.063 | | 0.714 | 0.223 | |
| 121437221 | | | | | | | | | | |
| chr12:133208886- | 94 | rs5745023 | rs5745022 | | | 0.134 | | 0.435 | 0.301 | |
| 133208979 | | | | | | | | | | |
| chr12:133226159- | 38 | rs4883613 | rs4883537 | | | 0.143 | | 0.271 | 0.414 | |
| 133226196 | | | | | | | | | | |
| chr12:133253995- | 89 | rs5744751 | rs5744750 | | | 0.057 | | 0.057 | 0.435 | |
| 133254083 | | | | | | | | | | |
| chr12:18656174- | 52 | rs11044141 | rs11044142 | | | 0.027 | | 0.134 | 0.161 | |
| 18656225 | | | | | | | | | | |
| chr12:56494991- | 8 | rs2271189 | rs773123 | | | 0.066 | | 0.252 | 0.067 | |
| 56494998 | | | | | | | | | | |
| chr13:21562832- | 117 | rs2770928 | rs558614 | | | 0.150 | | 0.150 | 0.370 | |
| 21562948 | | | | | | | | | | |
| chr14:102568296- | 72 | rs10873531 | rs8005905 | | | 0.137 | | 0.336 | 0.199 | |
| 102568367 | | | | | | | | | | |
| chr14:104165753- | 175 | rs861539 | rs1799796 | | | 0.217 | | 0.217 | 0.247 | |
| 104165927 | | | | | | | | | | |
| chr14:105239146- | 47 | rs3803304 | rs2494732 | | | 0.221 | | 0.221 | 0.426 | |
| 105239192 | | | | | | | | | | |
| chr14:105258892- | 2 | rs2494748 | rs2494749 | | | 0.291 | | 0.356 | 0.291 | |
| 105258893 | | | | | | | | | | |
| chr14:35872792- | 135 | rs2233415 | rs1050851 | | | 0.098 | | 0.333 | 0.102 | |
| 35872926 | | | | | | | | | | |
| chr15:40998305- | 38 | rs45592734 | rs45457497 | | | 0.204 | | 0.204 | 0.354 | |
| 40998342 | | | | | | | | | | |
| chr15:41857216- | 88 | rs11639399 | rs2277536 | | | 0.160 | | 0.160 | 0.267 | |
| 41857303 | | | | | | | | | | |
| chr15:41860411- | 80 | rs7171675 | rs12148316 | | | 0.154 | | 0.333 | 0.155 | |
| 41860490 | | | | | | | | | | |
| chr15:67457335- | 151 | rs1065080 | rs2289261 | | | 0.166 | | 0.166 | 0.485 | |
| 67457485 | | | | | | | | | | |
| chr16:2138269- | 130 | rs1748 | rs13332221 | | | 0.128 | 0.020 | 0.276 | 0.168 | |
| 2138398 | | | | | | | | | | |
| chr16:2138398- | 25 | rs13332221 | rs13332222 | | | 0.033 | | 0.168 | 0.201 | |
| 2138422 | | | | | | | | | | |
| chr16:68857289- | 153 | rs2276330 | rs1801552 | | | 0.058 | | 0.058 | 0.281 | |
| 68857441 | | | | | | | | | | |
| chr16:81819768- | 53 | rs1143685 | rs4294811 | | | 0.265 | | 0.267 | 0.286 | |
| 81819820 | | | | | | | | | | |
| chr16:89806343- | 5 | rs11647746 | rs7195906 | | | 0.141 | | 0.141 | 0.293 | |
| 89806347 | | | | | | | | | | |
| chr16:89849583- | 47 | rs2239360 | rs12448860 | | | 0.072 | | 0.387 | 0.324 | |
| 89849629 | | | | | | | | | | |
| chr16:89858505- | 21 | rs6500452 | rs1800287 | | | 0.172 | | 0.468 | 0.297 | |
| 89858525 | | | | | | | | | | |
| chr17:1782952- | 6 | rs5030755 | rs2230930 | | | 0.029 | | 0.029 | 0.271 | |
| 1782957 | | | | | | | | | | |
| chr17:78599562- | 94 | rs17848685 | rs901065 | | | ND | | Not in | 0.321 | |
| 78599655 | | | | | | | | 1 K | | |
| chr17:78820329- | 46 | rs3751945 | rs2589156 | | | 0.077 | | 0.437 | 0.077 | |
| 78820374 | | | | | | | | | | |
| chr17:78865546- | 85 | rs2289764 | rs2289765 | | | 0.161 | | 0.281 | 0.230 | |
| 78865630 | | | | | | | | | | |
| chr17:78897547- | 15 | rs7217786 | rs6565491 | | | 0.148 | | 0.249 | 0.148 | |
| 78897561 | | | | | | | | | | |
| chr17:78921117- | 95 | rs4969231 | rs9912373 | | | 0.119 | | 0.198 | 0.119 | |
| 78921211 | | | | | | | | | | |
| chr19:10267011- | 67 | rs4804490 | rs2228611 | | | 0.204 | | 0.204 | 0.466 | |
| 10267077 | | | | | | | | | | |
| chr19:17937758- | 29 | rs3212798 | rs3212797 | | | 0.028 | | 0.206 | 0.188 | |
| 17937786 | | | | | | | | | | |
| chr19:17955001- | 21 | rs3212713 | rs3212712 | rs3212711 | 17955003 | 0.051 | | 0.411 | 0.463 | 0.407 |
| 17955021 | | | | | | | | | | |
| chr19:2226676- | 97 | rs3815308 | rs2302061 | | | 0.225 | | 0.226 | 0.256 | |
| 2226772 | | | | | | | | | | |
| chr19:3119184- | 56 | rs308046 | rs4900 | | | 0.225 | | 0.226 | 0.349 | |
| 3119239 | | | | | | | | | | |
| chr19:50919797- | 32 | rs3218776 | rs3218760 | | | 0.278 | | 0.408 | 0.278 | |
| 50919828 | | | | | | | | | | |
| chr19:5210622- | 161 | rs2302224 | rs1143698 | | | 0.086 | 0.033 | 0.282 | 0.335 | |
| 5210782 | | | | | | | | | | |
| chr19:5210762- | 21 | rs1143699 | rs1143698 | | | 0.101 | | 0.101 | 0.335 | |
| 5210782 | | | | | | | | | | |
| chr19:5212380- | 103 | rs1064300 | rs2230611 | | | 0.144 | | 0.318 | 0.145 | |
| 5212482 | | | | | | | | | | |
| chr19:7166376- | 13 | rs2059806 | rs2229429 | | | 0.245 | | 0.245 | 0.257 | |
| 7166388 | | | | | | | | | | |
| chr2:112754828- | 53 | rs3811632 | rs3811633 | | | 0.190 | | 0.304 | 0.190 | |
| 112754880 | | | | | | | | | | |
| chr2:112754943- | 59 | rs3811634 | rs2230515 | | | 0.190 | | 0.191 | 0.439 | |
| 112755001 | | | | | | | | | | |
| chr2:141259283- | 94 | rs35296183 | rs35164907 | | | 0.022 | | 0.104 | 0.126 | |
| 141259376 | | | | | | | | | | |
| chr2:29416366- | 116 | rs1881421 | rs1881420 | | | 0.176 | 0.019 | 0.427 | 0.415 | |
| 29416481 | | | | | | | | | | |
| chr2:29416481- | 135 | rs1881420 | rs56132472 | | | 0.059 | | 0.415 | 0.059 | |
| 29416615 | | | | | | | | | | |
| chr2:29446184- | 19 | rs2276550 | rs4622670 | | | 0.177 | | 0.421 | 0.176 | |
| 29446202 | | | | | | | | | | |
| chr2:48010488- | 71 | rs1042821 | rs1042820 | | | 0.069 | | 0.201 | 0.069 | |
| 48010558 | | | | | | | | | | |
| chr20:40714307- | 173 | rs3092662 | rs2016647 | | | 0.062 | | 0.063 | 0.144 | |
| 40714479 | | | | | | | | | | |
| chr20:40714539- | 2 | rs1569547 | rs1569548 | | | 0.107 | | 0.108 | 0.244 | |
| 40714540 | | | | | | | | | | |
| chr20:57478807- | 133 | rs7121 | rs3730168 | | | 0.127 | 0.124 | 0.356 | 0.353 | |
| 57478939 | | | | | | | | | | |
| chr20:9543622- | 60 | rs2297345 | rs2297346 | | | 0.165 | | 0.485 | 0.350 | |
| 9543681 | | | | | | | | | | |
| chr21:42845374- | 10 | rs2298659 | rs17854725 | | | 0.151 | 0.059 | 0.209 | 0.366 | |
| 42845383 | | | | | | | | | | |
| chr22:21337266- | 60 | rs178280 | rs13054014 | | | 0.285 | | 0.357 | 0.285 | |
| 21337325 | | | | | | | | | | |
| chr22:21348914- | 124 | rs4822790 | rs178292 | | | 0.168 | | 0.169 | 0.248 | |
| 21349037 | | | | | | | | | | |
| chr22:24158895- | 5 | rs9608192 | rs2070457 | | | 0.105 | | 0.105 | 0.271 | |
| 24158899 | | | | | | | | | | |
| chr3:178922222- | 53 | rs3729676 | rs2699896 | | | 0.273 | | 0.273 | 0.415 | |
| 178922274 | | | | | | | | | | |
| chr3:183211906- | 121 | rs1520101 | rs2256061 | | | 0.151 | | 0.302 | 0.151 | |
| 183212026 | | | | | | | | | | |
| chr4:106196829- | 123 | rs34402524 | rs2454206 | | | 0.092 | | 0.092 | 0.230 | |
| 106196951 | | | | | | | | | | |
| chr4:143043340- | 65 | rs2270658 | rs13133767 | | | 0.101 | | 0.149 | 0.101 | |
| 143043404 | | | | | | | | | | |
| chr4:143324036- | 59 | rs1982965 | rs1982966 | | | 0.252 | | 0.454 | 0.253 | |
| 143324094 | | | | | | | | | | |
| chr4:187534362- | 14 | rs2249916 | rs2249917 | | | 0.194 | | 0.389 | 0.418 | |
| 187534375 | | | | | | | | | | |
| chr4:187629497- | 42 | rs458021 | rs3733413 | | | 0.084 | | 0.422 | 0.339 | |
| 187629538 | | | | | | | | | | |
| chr5:149456772- | 40 | rs60844779 | rs3829987 | | | 0.197 | | 0.310 | 0.197 | |
| 149456811 | | | | | | | | | | |
| chr5:149495287- | 109 | rs2229561 | rs246388 | | | ND | | Not in | 0.285 | |
| 149495395 | | | | | | | | 1 K | | |
| chr5:176517326- | 136 | rs422421 | rs446382 | | | 0.077 | | 0.147 | 0.224 | |
| 176517461 | | | | | | | | | | |
| chr5:176523562- | 36 | rs31777 | rs31776 | | | 0.068 | | 0.147 | 0.215 | |
| 176523597 | | | | | | | | | | |
| chr5:176721198- | 75 | rs28580074 | rs11740250 | | | 0.108 | | 0.229 | 0.108 | |
| 176721272 | | | | | | | | | | |
| chr5:180046209- | 136 | rs446003 | rs448012 | | | 0.070 | 0.021 | 0.368 | 0.417 | |
| 180046344 | | | | | | | | | | |
| chr5:180051003- | 116 | rs307826 | rs728986 | | | 0.053 | | 0.053 | 0.116 | |
| 180051118 | | | | | | | | | | |
| chr5:180057231- | 63 | rs3736061 | rs34221241 | | | 0.039 | | 0.059 | 0.039 | |
| 180057293 | | | | | | | | | | |
| chr5:231111- | 33 | rs1126417 | rs2288459 | | | 0.247 | | 0.347 | 0.247 | |
| 231143 | | | | | | | | | | |
| chr5:35861068- | 92 | rs1494558 | rs11567705 | rs969128 | 35861152 | 0.234 | 0.128 | 0.400 | 0.234 | 0.128 |
| 35861159 | | | | | | | | | | |
| chr5:35871190- | 84 | rs1494555 | rs2228141 | | | 0.129 | | 0.333 | 0.129 | |
| 35871273 | | | | | | | | | | |
| chr5:57754808- | 44 | rs697133 | rs702722 | | | 0.170 | | 0.260 | 0.170 | |
| 57754851 | | | | | | | | | | |
| chr5:67522722- | 130 | rs706713 | rs706714 | | | 0.035 | 0.029 | 0.419 | 0.425 | |
| 67522851 | | | | | | | | | | |
| chr6:117725448- | 131 | rs1998206 | rs2243378 | | | 0.168 | | 0.168 | 0.325 | |
| 117725578 | | | | | | | | | | |
| chr6:117730673- | 147 | rs17634067 | rs2273601 | | | 0.060 | | 0.059 | 0.360 | |
| 117730819 | | | | | | | | | | |
| chr6:152382311- | 15 | rs2273206 | rs2273207 | | | 0.115 | | 0.277 | 0.162 | |
| 152382325 | | | | | | | | | | |
| chr6:26056549- | 160 | rs10425 | rs2230653 | rs12204800 | 26056604 | 0.175 | 0.117 | 0.239 | 0.175 | 0.117 |
| 26056708 | | | | | | | | | | |
| chr6:30865115- | 90 | rs2239517 | rs2267641 | | | 0.125 | | 0.407 | 0.282 | |
| 30865204 | | | | | | | | | | |
| chr6:32188603- | 40 | rs520803 | rs520692 | rs520688 | 32188605 | 0.012 | | 0.268 | 0.268 | 0.280 |
| 32188642 | | | | | | | | | | |
| chr7:100410597- | 61 | rs2230585 | rs770657085 | | | 0.149 | | 0.276 | 0.424 | |
| 100410657 | | | | | | | | | | |
| chr7:6026775- | 168 | rs2228006 | rs1805323 | | | 0.112 | | 0.117 | 0.112 | |
| 6026942 | | | | | | | | | | |
| chr7:78119109- | 91 | rs3735442 | rs1990577 | | | ND | | 0.323 | Not in | |
| 78119199 | | | | | | | | | 1 K | |
| chr8:30999122- | 2 | rs3024239 | rs2737335 | | | 0.130 | | 0.375 | 0.495 | |
| 30999123 | | | | | | | | | | |
| chr8:31024638- | 17 | rs1801196 | rs1346044 | | | 0.193 | | 0.274 | 0.193 | |
| 31024654 | | | | | | | | | | |
| chr8:90958422- | 109 | rs1061302 | rs2308962 | | | 0.026 | | 0.353 | 0.379 | |
| 90958530 | | | | | | | | | | |
| chr9:139403268- | 13 | rs3125000 | rs11145765 | | | 0.088 | | 0.238 | 0.088 | |
| 139403280 | | | | | | | | | | |
| chr9:139405093- | 169 | rs36119806 | rs3125001 | | | 0.107 | | 0.108 | 0.414 | |
| 139405261 | | | | | | | | | | |
| chr9:139410424- | 166 | rs3125006 | rs4880099 | | | 0.115 | | 0.116 | 0.313 | |
| 139410589 | | | | | | | | | | |
| chr9:139411714- | 167 | rs11145767 | rs9411254 | | | 0.080 | | 0.395 | 0.474 | |
| 139411880 | | | | | | | | | | |
| chr9:21968159- | 41 | rs3088440 | rs11515 | | | 0.098 | | 0.170 | 0.098 | |
| 21968199 | | | | | | | | | | |
| chr9:93639846- | 128 | rs290223 | rs2290888 | | | ND | | Not in | 0.197 | |
| 93639973 | | | | | | | | 1 K | | |
| chr9:93641175- | 25 | rs2306041 | rs2306040 | | | 0.068 | | 0.198 | 0.131 | |
| 93641199 | | | | | | | | | | |
| chr9:98238358- | 22 | rs2066836 | rs1805155 | | | 0.092 | | 0.092 | 0.112 | |
| 98238379 | | | | | | | | | | |

TABLE 6
SBS sets for exome analysis.

| | | | Middle | | Middle | 3rd | 4th + | SNP1 | SNP2 | SNP3 |
|---|---|---|---|---|---|---|---|---|---|---|
| Location | Length | Start SNP | SNP | End SNP | Pos 1 | MH | MH | MAF | MAF | MAF |
| chr1:3743319- | 73 | rs6663840 | rs58111155 | rs6688969 | 4E+06 | 0.2 | 0.18 | 0.47 | 0.05 | 0.33 |
| 3743391 | | | | | | | | | | |
| chr1:10431132- | 27 | rs12141192 | | rs17411502 | | 0.14 | | 0.14 | | 0.25 |
| 10431158 | | | | | | | | | | |
| chr1:32672908- | 25 | rs3903683 | | rs12032332 | | 0.1 | | 0.23 | | 0.1 |
| 32672932 | | | | | | | | | | |
| chr1:94544234- | 43 | rs3112831 | | rs4147830 | | 0.22 | | 0.22 | | 0.49 |
| 94544276 | | | | | | | | | | |
| chr1:154832290- | 15 | rs1061122 | | rs4845397 | | 0.07 | | 0.22 | | 0.28 |
| 154832304 | | | | | | | | | | |
| chr1:159409857- | 28 | rs12048482 | | rs12118628 | | 0.13 | | 0.48 | | 0.13 |
| 159409884 | | | | | | | | | | |
| chr1:171168545- | 40 | rs2307492 | | rs2020862 | | 0.12 | | 0.12 | | 0.47 |
| 171168584 | | | | | | | | | | |
| chr1:183616884- | 43 | rs10911390 | | rs1174657 | | 0.09 | | 0.09 | | 0.37 |
| 183616926 | | | | | | | | | | |
| chr11:4928841- | 26 | rs7108225 | | rs7941509 | | 0.06 | | 0.06 | | 0.4 |
| 4928866 | | | | | | | | | | |
| chr11:5345128- | 43 | rs10837814 | | rs7952293 | | 0.24 | | 0.44 | | 0.24 |
| 5345170 | | | | | | | | | | |
| chr11:5566030- | 22 | rs1995158 | | rs1995157 | | 0.11 | | 0.11 | | 0.38 |
| 5566051 | | | | | | | | | | |
| chr11:63883985- | 43 | rs614397 | | rs614035 | | 0.12 | | 0.47 | | 0.41 |
| 63884027 | | | | | | | | | | |
| chr11:85436303- | 50 | rs3851177 | | rs641393 | | 0.09 | | 0.09 | | 0.48 |
| 85436352 | | | | | | | | | | |
| chr11:116703640- | 32 | rs5128 | | rs4225 | | 0.23 | | 0.23 | | 0.29 |
| 116703671 | | | | | | | | | | |
| chr12:6030405- | 33 | rs3741903 | | rs3741904 | | 0.07 | | 0.16 | | 0.1 |
| 6030437 | | | | | | | | | | |
| chr12:40834918- | 38 | rs4768261 | | rs10784618 | | 0.05 | | 0.05 | | 0.48 |
| 40834955 | | | | | | | | | | |
| chr12:113348849- | 22 | rs7955146 | | rs1131454 | | 0.1 | | 0.1 | | 0.47 |
| 113348870 | | | | | | | | | | |
| chr12:121600180- | 74 | rs208293 | | rs208294 | | 0.11 | 0.05 | 0.47 | | 0.47 |
| 121600253 | | | | | | | | | | |
| chr12:132688115- | 23 | rs11246991 | | rs7486927 | | 0.05 | | 0.05 | | 0.43 |
| 132688137 | | | | | | | | | | |
| chr13:25367282- | 20 | rs1451568 | | rs1158061 | | 0.16 | | 0.16 | | 0.25 |
| 25367301 | | | | | | | | | | |
| chr14:23549285- | 35 | rs3751501 | | rs1885097 | | 0.05 | | 0.05 | | 0.43 |
| 23549319 | | | | | | | | | | |
| chr14:65263300- | 48 | rs229587 | | rs229586 | | 0.19 | | 0.47 | | 0.28 |
| 65263347 | | | | | | | | | | |
| chr14:96136775- | 20 | rs2296310 | | rs2249778 | | 0.15 | | 0.18 | | 0.33 |
| 96136794 | | | | | | | | | | |
| chr15:41819283- | 40 | rs2297379 | | rs2297380 | | 0.31 | | 0.33 | | 0.31 |
| 41819322 | | | | | | | | | | |
| chr15:79310256- | 33 | rs16970441 | | rs2304994 | | 0.06 | | 0.06 | | 0.16 |
| 79310288 | | | | | | | | | | |
| chr15:89398330- | 78 | rs3743399 | | rs3743398 | | ND | | ND | | 0.08 |
| 89398407 | | | | | | | | | | |
| chr15:94945704- | 16 | rs7180682 | | rs7178698 | | 0.24 | | 0.24 | | 0.38 |
| 94945719 | | | | | | | | | | |
| chr16:2812890- | 50 | rs2240141 | | rs2240140 | | 0.26 | | 0.33 | | 0.41 |
| 2812939 | | | | | | | | | | |
| chr16:87678144- | 22 | rs918368 | | rs3751725 | | 0.19 | | 0.35 | | 0.19 |
| 87678165 | | | | | | | | | | |
| chr17:1782952- | 6 | rs5030755 | | rs2230930 | | 0.03 | | 0.03 | | 0.27 |
| 1782957 | | | | | | | | | | |
| chr17:3101578- | 13 | rs2241091 | | rs2469791 | | 0.15 | | 0.28 | | 0.15 |
| 3101590 | | | | | | | | | | |
| chr17:3352294- | 16 | rs1488689 | | rs11556563 | | 0.17 | | 0.27 | | 0.17 |
| 3352309 | | | | | | | | | | |
| chr17:6331803- | 34 | rs8075035 | | rs12453262 | | 0.09 | | 0.42 | | 0.49 |
| 6331836 | | | | | | | | | | |
| chr17:10223697- | 18 | rs2074876 | | rs2074877 | | 0.22 | | 0.24 | | 0.46 |
| 10223714 | | | | | | | | | | |
| chr17:33772658- | 32 | rs8072510 | | rs12943866 | | 0.07 | | 0.09 | | 0.07 |
| 33772689 | | | | | | | | | | |
| chr17:42989063- | 26 | rs1126642 | | rs2289681 | | 0.06 | | 0.06 | | 0.14 |
| 42989088 | | | | | | | | | | |
| chr17:45695832- | 83 | rs3760370 | | rs3760371 | | 0.08 | | 0.46 | | 0.38 |
| 45695914 | | | | | | | | | | |
| chr17:80887206- | 39 | rs729124 | | rs1127986 | | 0.23 | 0.01 | 0.32 | | 0.24 |
| 80887244 | | | | | | | | | | |
| chr18:56204747- | 22 | rs3826593 | | rs3809974 | | 0.06 | | 0.2 | | 0.06 |
| 56204768 | | | | | | | | | | |
| chr19:4510530- | 31 | rs7250947 | | rs7251858 | | 0.07 | | 0.07 | | 0.36 |
| 4510560 | | | | | | | | | | |
| chr19:8148301- | 14 | rs17202517 | | rs17160149 | | 0.12 | | 0.12 | | 0.32 |
| 8148314 | | | | | | | | | | |
| chr19:9362297- | 47 | rs12980833 | | rs2240927 | | 0.09 | | 0.09 | | 0.47 |
| 9362343 | | | | | | | | | | |
| chr19:11227554- | 49 | rs1799898 | | rs688 | | 0.09 | | 0.09 | | 0.28 |
| 11227602 | | | | | | | | | | |
| chr19:36237227- | 19 | rs3817622 | | rs2293688 | | 0.1 | | 0.1 | | 0.4 |
| 36237245 | | | | | | | | | | |
| chr19:44352639- | 28 | rs1061768 | rs2356437 | rs1061769 | 4E+07 | 0.15 | 0.15 | 0.15 | 0.32 | 0.39 |
| 44352666 | | | | | | | | | | |
| chr19:58131576- | 48 | rs10414451 | | rs10413455 | | 0.07 | | 0.07 | | 0.09 |
| 58131623 | | | | | | | | | | |
| chr19:58213952- | 18 | rs2074078 | | rs11878316 | | 0.14 | | 0.17 | | 0.14 |
| 58213969 | | | | | | | | | | |
| chr19:58572959- | 21 | rs2288274 | | rs1469087 | | 0.22 | | 0.27 | | 0.22 |
| 58572979 | | | | | | | | | | |
| CHR2:33623720- | 15 | rs8970 | | rs622716 | | 0.22 | | 0.31 | | 0.22 |
| 33623734 | | | | | | | | | | |
| CHR2:37579937- | 35 | rs2302652 | | rs2255991 | | 0.14 | | 0.29 | | 0.14 |
| 37579971 | | | | | | | | | | |
| CHR2:71058184- | 43 | rs13421115 | | rs2080390 | | 0.14 | | 0.16 | | 0.14 |
| 71058226 | | | | | | | | | | |
| CHR2:231775094- | 51 | rs3749073 | | rs1992187 | | 0.05 | | 0.2 | | 0.05 |
| 231775144 | | | | | | | | | | |
| CHR2:239184569- | 13 | rs13391269 | | rs10462023 | | 0.07 | | 0.07 | | 0.23 |
| 239184581 | | | | | | | | | | |
| chr20:744382- | 34 | rs3746803 | | rs3746804 | | 0.09 | | 0.09 | | 0.18 |
| 744415 | | | | | | | | | | |
| chr20:5904028- | 13 | rs742710 | | rs742711 | | 0.18 | | 0.18 | | 0.23 |
| 5904040 | | | | | | | | | | |
| chr20:52645534- | 8 | rs466264 | | rs2072127 | | 0.05 | | 0.3 | | 0.05 |
| 52645541 | | | | | | | | | | |
| chr20:62597666- | 29 | rs45486695 | | rs817329 | | 0.07 | | 0.07 | | 0.49 |
| 62597694 | | | | | | | | | | |
| chr21:43557698- | 39 | rs3819142 | | rs220178 | | 0.22 | | 0.22 | | 0.29 |
| 43557736 | | | | | | | | | | |
| chr21:46321659- | 19 | rs55865320 | | rs5030669 | | 0.12 | | 0.14 | | 0.12 |
| 46321677 | | | | | | | | | | |
| chr22:17589209- | 38 | rs879577 | | rs879576 | | 0.12 | | 0.27 | | 0.12 |
| 17589246 | | | | | | | | | | |
| chr22:19951207- | 65 | rs4818 | | rs4680 | | 0.3 | | 0.3 | | 0.37 |
| 19951271 | | | | | | | | | | |
| chr22:21377301- | 34 | rs1548411 | | rs1548412 | | 0.17 | | 0.37 | | 0.17 |
| 21377334 | | | | | | | | | | |
| chr22:33253280- | 13 | rs9862 | | rs11547635 | | 0.14 | | 0.35 | | 0.14 |
| 33253292 | | | | | | | | | | |
| chr22:35817553- | 45 | rs2071744 | | rs133431 | | 0.16 | | 0.16 | | 0.45 |
| 35817597 | | | | | | | | | | |
| chr22:44322922- | 49 | rs2076213 | | rs2076212 | | 0.04 | 0.04 | 0.07 | | 0.12 |
| 44322970 | | | | | | | | | | |
| chr3:122003757- | 13 | rs1801725 | | rs1042636 | | 0.09 | | 0.09 | | 0.21 |
| 122003769 | | | | | | | | | | |
| chr3:129155451- | 13 | rs140693 | | rs2307289 | | 0.07 | | 0.11 | | 0.07 |
| 129155463 | | | | | | | | | | |
| chr3:136574501- | 21 | rs1052618 | | rs1052620 | | 0.09 | | 0.29 | | 0.09 |
| 136574521 | | | | | | | | | | |
| chr3:142277536- | 40 | rs2227929 | | rs2227930 | | 0.29 | | 0.31 | | 0.4 |
| 142277575 | | | | | | | | | | |
| chr3:178968634- | 27 | rs7645550 | | rs1170672 | | 0.07 | | 0.32 | | 0.07 |
| 178968660 | | | | | | | | | | |
| chr4:156289900- | 18 | rs3733390 | | rs3733391 | | 0.17 | | 0.37 | | 0.17 |
| 156289917 | | | | | | | | | | |
| chr5:147024476- | 34 | rs2116766 | | rs2116765 | | ND | | ND | | 0.37 |
| 147024509 | | | | | | | | | | |
| chr5:148206440- | 34 | rs1042713 | | rs1042714 | | 0.2 | | 0.48 | | 0.2 |
| 148206473 | | | | | | | | | | |
| chr5:150666933- | 30 | rs375396 | | rs12520516 | | 0.1 | | 0.25 | | 0.1 |
| 150666962 | | | | | | | | | | |
| chr5:150901613- | 18 | rs2053028 | | rs3734049 | | 0.1 | | 0.22 | | 0.1 |
| 150901630 | | | | | | | | | | |
| chr5:174870150- | 47 | rs4532 | | rs5326 | | 0.17 | | 0.25 | | 0.17 |
| 174870196 | | | | | | | | | | |
| chr6:4069133- | 34 | rs10485172 | | rs595413 | | ND | | ND | | 0.45 |
| 4069166 | | | | | | | | | | |
| chr6:29913201- | 66 | rs41557912 | | rs1061156 | | 0.15 | | 0.15 | | 0.2 |
| 29913266 | | | | | | | | | | |
| chr6:30080231- | 44 | rs3734838 | | rs2517598 | | 0.07 | | 0.07 | | 0.12 |
| 30080274 | | | | | | | | | | |
| chr6:30993533- | 58 | rs2523898 | rs4713420 | rs12179536 | 3E+07 | 0.13 | 0.25 | 0.44 | 0.21 | 0.2 |
| 30993590 | | | | | | | | | | |
| chr6:31170514- | 15 | rs9263870 | | rs9263871 | | 0.13 | | 0.13 | | 0.38 |
| 31170528 | | | | | | | | | | |
| chr6:31930441- | 22 | rs592229 | | rs429608 | | 0.15 | | 0.35 | | 0.15 |
| 31930462 | | | | | | | | | | |
| chr6:33141253- | 28 | rs9277932 | | rs2855430 | | 0.1 | | 0.36 | | 0.1 |
| 33141280 | | | | | | | | | | |
| chr6:36291985- | 23 | rs7751919 | | rs7751928 | | 0.11 | | 0.11 | | 0.28 |
| 36292007 | | | | | | | | | | |
| chr6:167754702- | 20 | rs909546 | | rs9457304 | | 0.06 | | 0.49 | | 0.06 |
| 167754721 | | | | | | | | | | |
| chr7:4213975- | 49 | rs671694 | | rs886731 | | 0.07 | 0.02 | 0.2 | | 0.09 |
| 4214023 | | | | | | | | | | |
| chr7:21640361- | 45 | rs10269582 | | rs10224537 | | 0.22 | | 0.22 | | 0.23 |
| 21640405 | | | | | | | | | | |
| chr7:27196069- | 45 | rs2301720 | | rs2301721 | | 0.15 | | 0.23 | | 0.38 |
| 27196113 | | | | | | | | | | |
| chr7:30795288- | 44 | rs2302339 | | rs2302340 | | 0.25 | | 0.25 | | 0.33 |
| 30795331 | | | | | | | | | | |
| chr7:55220177- | 26 | rs11506105 | | rs845561 | | 0.21 | 0.17 | 0.45 | | |
| 55220202 | | | | | | | | | | |
| chr7:100677455- | 69 | rs61075804 | | rs10238201 | | 0.04 | 0.02 | 0.2 | | 0.18 |
| 100677523 | | | | | | | | | | |
| CHR8:142490120- | 47 | rs2748416 | | rs7838192 | | 0.16 | | 0.22 | | 0.16 |
| 142490166 | | | | | | | | | | |
| CHR8:145639681- | 46 | rs1871534 | | rs2272662 | | 0.24 | | 0.25 | | 0.39 |
| 145639726 | | | | | | | | | | |
| chr9:117166206- | 41 | rs2274158 | | rs2274159 | | 0.18 | | 0.22 | | 0.41 |
| 117166246 | | | | | | | | | | |
| chr9:125315542- | 16 | rs1831369 | | rs1831370 | | 0.18 | | 0.38 | | 0.44 |
| 125315557 | | | | | | | | | | |
| chr9:134385435- | 2 | rs3887873 | | rs2296949 | | 0.08 | | 0.08 | | 0.13 |
| 134385436 | | | | | | | | | | |
| chr9:136412255- | 42 | rs2073876 | | rs2073877 | | 0.1 | | 0.28 | | 0.1 |
| 136412296 | | | | | | | | | | |
| chrX:23019317- | 30 | rs5925720 | | rs5926203 | | 0.16 | | 0.16 | | 0.34 |
| 23019346 | | | | | | | | | | |

TABLE 7
SNP sets.

| | | | | Medi- | | | | | | | | | | |
|---|---|---|---|---|---|---|---|---|---|---|---|---|---|---|
| | | | | an | | | | | | | | | Ad- | |
| | 1^{ST} | 2^{ND} | | Pan- | Pure, | | | | | Afri- | East | Euro- | mix | South |
| | Pan- | Pan- | | el | MH > | | | | | can | Asian | pean | Amer | Asian |
| Location | el | el | Exome | Cov | 2 | Length | SNP1 | SNP2 | SNP3 | 3 + 4 | 3 + 4 | 3 + 4 | 3 + 4 | 3 + 4 |
| chr1:10431132- | | | Yes | 0 | 0 | 27 | rs12141192 | rs17411502 | | | | | | |
| 10431158 | | | | | | | | | | | | | | |
| chr1:120057158- | Yes | | | 689 | 3 | 89 | rs6203 | rs45609334 | | 0.033 | 0.082 | 0.235 | | |
| 120057246 | | | | | | | | | | | | | | |
| chr1:154832290- | | | Yes | 0 | 0 | 15 | rs1061122 | rs4845397 | | | | | | |
| 154832304 | | | | | | | | | | | | | | |
| chr1:156846120- | Yes | Yes | | 1526 | 2 | 114 | rs1800880 | rs6334 | | 0.105 | 0.139 | 0.065 | 0.117 | 0.24 [symbol] |
| 156846233 | | | | | | | | | | | | | | |
| chr1:159409857- | | | Yes | 0 | 0 | 28 | rs12048482 | rs12118628 | | | | | | |
| 159409884 | | | | | | | | | | | | | | |
| chr1:171168545- | | | Yes | 0 | 0 | 40 | rs2307492 | rs2020862 | | | | | | |
| 171168584 | | | | | | | | | | | | | | |
| chr1:183616884- | | | Yes | 0 | 0 | 43 | rs10911390 | rs1174657 | | | | | | |
| 183616926 | | | | | | | | | | | | | | |
| chr1:226573364- | | Yes | | 2011 | 1 | 39 | rs1805414 | rs1805408 | | 0.143 | 0.205 | 0.159 | 0.147 | 0.183 |
| 226573402 | | | | | | | | | | | | | | |
| chr1:226589833- | Yes | Yes | | 361 | 2 | 126 | rs1805407 | rs1805404 | | 0.115 | 0.251 | 0.154 | 0.147 | 0.100 |
| 226589958 | | | | | | | | | | | | | | |
| chr1:23885498- | Yes | | | 692 | 25 | 102 | rs11574 | rs2067053 | | 0.011 | 0.028 | 0.242 | | |
| 23885599 | | | | | | | | | | | | | | |
| chr1:32672908- | | | Yes | 0 | 0 | 25 | rs3903683 | rs12032332 | | | | | | |
| 32672932 | | | | | | | | | | | | | | |
| chr1:3743319- | | | Yes | 0 | 0 | 73 | rs6663840 | rs58111155 | rs6688969 | | | | | |
| 3743391 | | | | | | | | | | | | | | |
| chr1:94544234- | | | Yes | 0 | 0 | 43 | rs3112831 | rs4147830 | | | | | | |
| 94544276 | | | | | | | | | | | | | | |
| chr10:104386934- | Yes | Yes | | 250 | 0 | 86 | rs17114803 | rs12414407 | | 0.224 | 0.250 | 0.093 | 0.238 | 0.240 |
| 104387019 | | | | | | | | | | | | | | |
| chr10:123194558- | | Yes | | 384 | 0 | 52 | rs7911440 | rs6585731 | | 0.051 | 0.211 | 0.242 | 0.082 | 0.243 |
| 123194609 | | | | | | | | | | | | | | |
| chr10:123199092- | | Yes | | 1151 | 2 | 4 | rs4752560 | rs2114689 | | 0.283 | 0.023 | 0.075 | 0.156 | 0.160 |
| 123199095 | | | | | | | | | | | | | | |
| chr10:123275662- | | Yes | | 320 | 1 | 5 | rs2912761 | rs2981453 | | 0.211 | 0.000 | 0.000 | 0.050 | 0.000 |
| 123275666 | | | | | | | | | | | | | | |
| chr10:123335839- | | Yes | | 1055 | 1 | 28 | rs45631611 | rs10886946 | | 0.017 | 0.113 | 0.071 | 0.055 | 0.114 |
| 123335866 | | | | | | | | | | | | | | |
| chr10:123346116- | | Yes | | 420 | 0 | 75 | rs2981575 | rs1219648 | | 0.195 | 0.048 | 0.000 | 0.022 | 0.013 |
| 123346190 | | | | | | | | | | | | | | |
| chr10:123396728- | | Yes | | 331 | 2 | 79 | rs1909670 | rs1614303 | | 0.029 | 0.176 | 0.100 | 0.131 | 0.073 |
| 123396806 | | | | | | | | | | | | | | |
| chr10:123406645- | | Yes | | 699 | 4 | 19 | rs10788194 | rs7923788 | | 0.084 | 0.227 | 0.151 | 0.192 | 0.125 |
| 123406663 | | | | | | | | | | | | | | |
| chr10:43611708- | | Yes | | 629 | 2 | 158 | rs741968 | rs2256550 | | 0.060 | 0.218 | 0.161 | 0.212 | 0.284 |
| 43611865 | | | | | | | | | | | | | | |
| chr10:43615505- | Yes | Yes | | 463 | 5 | 129 | rs2472737 | rs1800863 | | 0.105 | 0.121 | 0.193 | 0.187 | 0.160 |
| 43615633 | | | | | | | | | | | | | | |
| chr10:70332580- | Yes | Yes | | 549 | 1 | 93 | rs10823229 | rs12773594 | | 0.023 | 0.173 | 0.185 | 0.151 | 0.271 |
| 70332672 | | | | | | | | | | | | | | |
| chr11:116703640- | | | Yes | 0 | 0 | 32 | rs5128 | rs4225 | | | | | | |
| 116703671 | | | | | | | | | | | | | | |
| chr11:4928841- | | | Yes | 0 | 0 | 26 | rs7108225 | rs7941509 | | | | | | |
| 4928866 | | | | | | | | | | | | | | |
| chr11:534197- | Yes | Yes | | 2026 | 1 | 46 | rs41258054 | rs12628 | | 0.000 | 0.153 | 0.056 | 0.137 | 0.076 |
| 534242 | | | | | | | | | | | | | | |
| chr11:5345128- | | | Yes | 0 | 0 | 43 | rs10837814 | rs7952293 | | | | | | |
| 5345170 | | | | | | | | | | | | | | |
| chr11:5566030- | | | Yes | 0 | 0 | 22 | rs1995158 | rs1995157 | | | | | | |
| 5566051 | | | | | | | | | | | | | | |
| chr11:63883985- | | | Yes | 0 | 0 | 43 | rs614397 | rs614035 | | | | | | |
| 63884027 | | | | | | | | | | | | | | |
| chr11:69412090- | | Yes | | 2968 | 1 | 35 | rs79274134 | rs7112989 | | 0.254 | 0.232 | 0.000 | 0.127 | 0.031 |
| 69412124 | | | | | | | | | | | | | | |
| chr11:8246326- | Yes | | | 287 | 6 | 18 | rs34544683 | rs3816490 | | 0.022 | 0.098 | 0.125 | | |
| 8246343 | | | | | | | | | | | | | | |
| chr11:85436303- | | | Yes | 0 | 0 | 50 | rs3851177 | rs641393 | | | | | | |
| 85436352 | | | | | | | | | | | | | | |
| chr12:113348849- | | | Yes | 0 | 0 | 22 | rs7955146 | rs1131454 | | | | | | |
| 113348870 | | | | | | | | | | | | | | |
| chr12:12009741- | | Yes | | 379 | 2 | 134 | rs2238126 | rs743614 | | 0.181 | 0.240 | 0.190 | 0.249 | 0.079 |
| 12009874 | | | | | | | | | | | | | | |
| chr12:12013572- | | Yes | | 647 | 3 | 41 | rs2855708 | rs6488463 | | 0.232 | 0.196 | 0.211 | 0.347 | 0.146 |
| 12013612 | | | | | | | | | | | | | | |
| chr12:12016008- | | Yes | | 1488 | 3 | 82 | rs2238130 | rs2416944 | rs2238131 | 0.125 | 0.248 | 0.144 | 0.216 | 0.104 |
| 12016089 | | | | | | | | | | | | | | |
| chr12:12020114- | | Yes | | 637 | 1 | 57 | rs2723805 | rs7973930 | | 0.241 | 0.111 | 0.075 | 0.066 | 0.054 |
| 12020170 | | | | | | | | | | | | | | |
| chr12:12035649- | | Yes | | 2052 | 1 | 16 | rs2710310 | rs2739085 | | 0.126 | 0.271 | 0.194 | 0.251 | 0.159 |
| 12035664 | | | | | | | | | | | | | | |
| chr12:121416622- | Yes | Yes | | 3076 | 2 | 29 | rs1169289 | rs1169288 | | 0.082 | 0.049 | 0.132 | 0.112 | 0.151 |
| 121416650 | | | | | | | | | | | | | | |
| chr12:121431272- | Yes | Yes | | 1774 | 0 | 29 | rs2071190 | rs1169301 | | 0.118 | 0.255 | 0.236 | 0.272 | 0.182 |
| 121431300 | | | | | | | | | | | | | | |
| chr12:121435427- | Yes | | | 3503 | 1 | 49 | rs2464196 | rs2464195 | | 0.014 | 0.000 | 0.062 | | |
| 121435475 | | | | | | | | | | | | | | |
| chr12:121437114- | Yes | | | 1919 | 0 | 108 | rs55834942 | rs1169304 | | 0.012 | 0.000 | 0.166 | | |
| 121437221 | | | | | | | | | | | | | | |
| chr12:121600180- | | | Yes | 0 | 0 | 74 | rs208293 | rs208294 | | | | | | |
| 121600253 | | | | | | | | | | | | | | |
| chr12:132688115- | | | Yes | 0 | 0 | 23 | rs11246991 | rs7486927 | | | | | | |
| 132688137 | | | | | | | | | | | | | | |
| chr12:133208886- | Yes | Yes | | 739 | 2 | 94 | rs5745023 | rs5745022 | | 0.173 | 0.105 | 0.135 | 0.219 | 0.049 |
| 133208979 | | | | | | | | | | | | | | |
| chr12:133226159- | Yes | Yes | | 587 | 2 | 38 | rs4883613 | rs4883537 | | 0.105 | 0.107 | 0.135 | 0.222 | 0.050 |
| 133226196 | | | | | | | | | | | | | | |
| chr12:133253995- | Yes | Yes | | 448 | 1 | 89 | rs5744751 | rs5744750 | | 0.000 | 0.105 | 0.100 | 0.045 | 0.042 |
| 133254083 | | | | | | | | | | | | | | |
| chr12:18656174- | Yes | | | 381 | 1 | 52 | rs11044141 | rs11044142 | | 0.099 | 0.000 | 0.000 | 0.000 | 0.000 |
| 18656225 | | | | | | | | | | | | | | |
| chr12:40834918- | | | Yes | 0 | 0 | 38 | rs4768261 | rs10784618 | | | | | | |
| 40834955 | | | | | | | | | | | | | | |
| chr12:4346169- | | Yes | | 646 | 0 | 9 | rs11063052 | rs11832328 | | 0.318 | 0.079 | 0.038 | 0.072 | 0.080 |
| 4346177 | | | | | | | | | | | | | | |
| chr12:4351884- | | Yes | | 468 | 5 | 144 | rs7955545 | rs4766223 | | 0.051 | 0.113 | 0.033 | 0.076 | 0.092 |
| 4352027 | | | | | | | | | | | | | | |
| chr12:4376089- | | Yes | | 306 | 2 | 3 | rs4238013 | rs12818766 | | 0.119 | 0.033 | 0.181 | 0.161 | 0.147 |
| 4376091 | | | | | | | | | | | | | | |
| chr12:4399036- | | Yes | | 1619 | 2 | 52 | rs3217859 | rs3217860 | rs3217861 | 0.325 | 0.391 | 0.414 | 0.491 | 0.479 |
| 4399087 | | | | | | | | | | | | | | |
| chr12:4399917- | | Yes | | 892 | 2 | 54 | rs3217867 | rs3217868 | rs3217869 | 0.173 | 0.041 | 0.220 | 0.133 | 0.188 |
| 4399970 | | | | | | | | | | | | | | |
| chr12:4411639- | | Yes | | 1376 | 1 | 45 | rs3217925 | rs3217926 | | 0.127 | 0.068 | 0.253 | 0.172 | 0.227 |
| 4411683 | | | | | | | | | | | | | | |
| chr12:4417127- | | Yes | | 1224 | 1 | 106 | rs7133323 | rs9668504 | | 0.449 | 0.324 | 0.237 | 0.282 | 0.142 |
| 4417232 | | | | | | | | | | | | | | |
| chr12:56494991- | Yes | | | 3387 | 6 | 8 | rs2271189 | rs773123 | | 0.073 | 0.000 | 0.110 | 0.066 | 0.070 |
| 56494998 | | | | | | | | | | | | | | |
| chr12:6030405- | | | Yes | 0 | 0 | 33 | rs3741903 | rs3741904 | | | | | | |
| 6030437 | | | | | | | | | | | | | | |
| chr12:69169222- | | Yes | | 404 | 3 | 95 | rs6581833 | rs73334654 | | 0.256 | 0.016 | 0.059 | 0.078 | 0.000 |
| 69169316 | | | | | | | | | | | | | | |
| chr12:69265196- | | Yes | | 768 | 0 | 83 | rs3817605 | rs2293637 | | 0.310 | 0.192 | 0.022 | 0.111 | 0.106 |
| 69265278 | | | | | | | | | | | | | | |
| chr12:69277127- | | Yes | | 773 | 1 | 39 | rs10878875 | rs1663588 | | 0.126 | 0.162 | 0.124 | 0.133 | 0.215 |
| 69277165 | | | | | | | | | | | | | | |
| chr13:21562832- | Yes | | | 1715 | 3 | 117 | rs2770928 | rs558614 | | 0.175 | 0.000 | 0.080 | 0.087 | 0.153 |
| 21562948 | | | | | | | | | | | | | | |
| chr13:25367282- | | | Yes | 0 | 0 | 20 | rs1451568 | rs1158061 | | | | | | |
| 25367301 | | | | | | | | | | | | | | |
| chr13:32986219- | | Yes | | 313 | 0 | | rs206319 | rs206320 | rs615762 | 0.107 | 0.204 | 0.175 | 0.244 | 0.262 |
| 32986340 | | | | | | | | | | | | | | |
| chr14:102568296- | Yes | | | 969 | 0 | 72 | rs10873531 | rs8005905 | | 0.278 | 0.049 | 0.017 | 0.068 | 0.123 |
| 102568367 | | | | | | | | | | | | | | |
| chr14:104165753- | Yes | | | 765 | 4 | 175 | rs861539 | rs1799796 | | 0.114 | 0.073 | 0.295 | | |
| 104165927 | | | | | | | | | | | | | | |
| chr14:105239146- | Yes | Yes | | 521 | 5 | 47 | rs3803304 | rs2494732 | | 0.169 | 0.097 | 0.171 | 0.290 | 0.302 |
| 105239192 | | | | | | | | | | | | | | |
| chr14:105258892- | Yes | Yes | | 737 | 1 | 2 | rs2494748 | rs2494749 | | 0.120 | 0.122 | 0.092 | 0.231 | 0.245 |
| 105258893 | | | | | | | | | | | | | | |
| chr14:23549285- | | | Yes | 0 | 0 | 35 | rs3751501 | rs1885097 | | | | | | |
| 23549319 | | | | | | | | | | | | | | |
| chr14:35872792- | Yes | | | 643 | 1 | 135 | rs2233415 | rs1050851 | | 0.020 | 0.019 | 0.213 | | |
| 35872926 | | | | | | | | | | | | | | |
| chr14:65263300- | | | Yes | 0 | 0 | 48 | rs229587 | rs229586 | | | | | | |
| 65263347 | | | | | | | | | | | | | | |
| chr14:96136775- | | | Yes | 0 | 0 | 20 | rs2296310 | rs2249778 | | | | | | |
| 96136794 | | | | | | | | | | | | | | |
| chr15:40998305- | Yes | | | 215 | 0 | 38 | rs45592734 | rs45457497 | | 0.070 | 0.112 | 0.153 | | |
| 40998342 | | | | | | | | | | | | | | |
| chr15:41819283- | | | Yes | 0 | 0 | 40 | rs2297379 | rs2297380 | | | | | | |
| 41819322 | | | | | | | | | | | | | | |
| chr15:41857216- | Yes | | | 1528 | 2 | 88 | rs11639399 | rs2277536 | | 0.096 | 0.012 | 0.308 | | |
| 41857303 | | | | | | | | | | | | | | |
| chr15:41860411- | Yes | | | 860 | 2 | 80 | rs7171675 | rs12148316 | | 0.095 | 0.011 | 0.134 | | |
| 41860490 | | | | | | | | | | | | | | |
| chr15:67457335- | Yes | Yes | | 475 | 4 | 151 | rs1065080 | rs2289261 | | 0.133 | 0.238 | 0.139 | 0.087 | 0.220 |
| 67457485 | | | | | | | | | | | | | | |
| chr15:79310256- | | | Yes | 0 | 0 | 33 | rs16970441 | rs2304994 | | | | | | |
| 79310288 | | | | | | | | | | | | | | |
| chr15:88488326- | | Yes | | 1800 | 1 | | rs8042993 | rs1369426 | | 0.088 | 0.135 | 0.153 | 0.097 | 0.261 |
| 88488428 | | | | | | | | | | | | | | |
| chr15:88549118- | | Yes | | 1763 | 0 | | rs11073758 | rs12324332 | | 0.266 | 0.015 | 0.124 | 0.133 | 0.079 |
| 88549151 | | | | | | | | | | | | | | |
| chr15:88646922- | | Yes | | 975 | 1 | | rs16941255 | rs76506232 | | 0.110 | 0.132 | 0.000 | 0.010 | 0.000 |
| 88647038 | | | | | | | | | | | | | | |
| chr15:88667852- | | Yes | | 1099 | 0 | | rs3784411 | rs3784410 | | 0.192 | 0.100 | 0.217 | 0.225 | 0.151 |
| 88667948 | | | | | | | | | | | | | | |
| chr15:89398330- | | | Yes | 0 | 0 | 78 | rs3743399 | rs3743398 | | ND | ND | ND | ND | ND |
| 89398407 | | | | | | | | | | | | | | |
| chr15:94945704- | | | Yes | 0 | 0 | 16 | rs7180682 | rs7178698 | | | | | | |
| 94945719 | | | | | | | | | | | | | | |
| chr16:2138269- | Yes | | | 941 | 4 | 130 | rs1748 | rs13332221 | | 0.249 | 0.000 | 0.116 | 0.017 | 0.123 |
| 2138398 | | | | | | | | | | | | | | |
| chr16:2138398- | Yes | Yes | | 2026 | 0 | 25 | rs13332221 | rs13332222 | | 0.118 | 0.000 | 0.000 | 0.013 | 0.000 |
| 2138422 | | | | | | | | | | | | | | |
| chr16:2812890- | | | Yes | 0 | 0 | 50 | rs2240141 | rs2240140 | | | | | | |
| 2812939 | | | | | | | | | | | | | | |
| chr16:68857289- | Yes | | | 215 | 1 | 153 | rs2276330 | rs1801552 | | 0.000 | 0.068 | 0.120 | 0.056 | 0.051 |
| 68857441 | | | | | | | | | | | | | | |
| chr16:81819768- | Yes | Yes | | 2558 | 1 | 53 | rs1143685 | rs4294811 | | 0.140 | 0.141 | 0.282 | 0.271 | 0.126 |
| 81819820 | | | | | | | | | | | | | | |
| chr16:87678144- | | | Yes | 0 | 0 | 22 | rs918368 | rs3751725 | | | | | | |
| 87678165 | | | | | | | | | | | | | | |
| chr16:89806343- | Yes | Yes | | 601 | 2 | 5 | rs11647746 | rs7195906 | | 0.161 | 0.013 | 0.074 | 0.035 | 0.134 |
| 89806347 | | | | | | | | | | | | | | |
| chr16:89849480- | Yes | | | 275 | 2 | 150 | rs2239359 | rs12448860 | | 0.032 | 0.013 | 0.064 | | |
| 89849629 | | | | | | | | | | | | | | |
| chr16:89858505- | Yes | | | 698 | 3 | 21 | rs6500452 | rs1800287 | | 0.177 | 0.012 | 0.073 | 0.043 | 0.133 |
| 89858525 | | | | | | | | | | | | | | |
| chr17:1782952- | Yes | Yes | Yes | 1284 | 1 | 6 | rs5030755 | rs2230930 | | 0.000 | 0.000 | 0.102 | 0.020 | 0.024 |
| 1782957 | | | | | | | | | | | | | | |
| chr17:3101578- | | | Yes | 0 | 0 | 13 | rs2241091 | rs2469791 | | | | | | |
| 3101590 | | | | | | | | | | | | | | |
| chr17:33772658- | | | Yes | 0 | 0 | 32 | rs8072510 | rs12943866 | | | | | | |
| 33772689 | | | | | | | | | | | | | | |
| chr17:37832279- | | Yes | | 1408 | 1 | 37 | rs1495100 | rs2934953 | | 0.194 | 0.000 | 0.016 | 0.062 | 0.053 |
| 37832315 | | | | | | | | | | | | | | |
| chr17:37834715- | | Yes | | 1558 | 5 | 94 | rs12150603 | rs72832915 | | 0.042 | 0.153 | 0.308 | 0.196 | 0.235 |
| 37834808 | | | | | | | | | | | | | | |
| chr17:41616392- | | Yes | | 1646 | 1 | | rs76280498 | rs7222604 | | 0.000 | 0.150 | 0.106 | 0.110 | 0.181 |
| 41616456 | | | | | | | | | | | | | | |
| chr17:42989063- | | | Yes | 0 | 0 | 26 | rs1126642 | rs2289681 | | | | | | |
| 42989088 | | | | | | | | | | | | | | |
| chr17:45695832- | | | Yes | 0 | 0 | 83 | rs3760370 | rs3760371 | | | | | | |
| 45695914 | | | | | | | | | | | | | | |
| chr17:6331803- | | | Yes | 0 | 0 | 34 | rs8075035 | rs12453262 | | | | | | |
| 6331836 | | | | | | | | | | | | | | |
| chr17:78599562- | Yes | | | 2120 | 0 | 94 | rs17848685 | rs901065 | | ND | ND | ND | ND | ND |
| 78599655 | | | | | | | | | | | | | | |
| chr17:78820329- | Yes | Yes | | 3252 | 0 | 46 | rs3751945 | rs2589156 | | 0.082 | 0.000 | 0.107 | 0.078 | 0.115 |
| 78820374 | | | | | | | | | | | | | | |
| chr17:78865546- | Yes | Yes | | 631 | 3 | 85 | rs2289764 | rs2289765 | | 0.289 | 0.044 | 0.111 | 0.110 | 0.115 |
| 78865630 | | | | | | | | | | | | | | |
| chr17:78896488- | | Yes | | 2726 | 4 | 42 | rs2271602 | rs2271603 | | 0.154 | 0.196 | 0.321 | 0.291 | 0.307 |
| 78896529 | | | | | | | | | | | | | | |
| chr17:78897547- | Yes | Yes | | 1725 | 0 | 15 | rs7217786 | rs6565491 | | 0.031 | 0.199 | 0.122 | 0.111 | 0.249 |
| 78897561 | | | | | | | | | | | | | | |
| chr17:78921117- | Yes | Yes | | 1576 | 2 | 95 | rs4969231 | rs9912373 | | 0.022 | 0.079 | 0.124 | 0.114 | 0.060 |
| 78921211 | | | | | | | | | | | | | | |
| chr17:80887206- | | | Yes | 0 | 0 | 39 | rs729124 | rs1127986 | | | | | | |
| 80887244 | | | | | | | | | | | | | | |
| chr18:56204747- | | | Yes | 0 | 0 | 22 | rs3826593 | rs3809974 | | | | | | |
| 56204768 | | | | | | | | | | | | | | |
| chr19:10267011- | Yes | Yes | | 265 | 0 | 67 | rs4804490 | rs2228611 | | 0.171 | 0.281 | 0.068 | 0.184 | 0.224 |
| 10267077 | | | | | | | | | | | | | | |
| chr19:11227554- | | | Yes | 0 | 0 | 49 | rs1799898 | rs688 | | | | | | |
| 11227602 | | | | | | | | | | | | | | |
| chr19:17937758- | Yes | | | 1721 | 0 | 29 | rs3212798 | rs3212797 | | 0.074 | 0.000 | 0.052 | | |
| 17937786 | | | | | | | | | | | | | | |
| chr19:17955001- | Yes | Yes | | 1946 | 1 | 21 | rs3212713 | rs3212712 | rs3212711 | 0.197 | 0.000 | 0.000 | 0.022 | 0.000 |
| 17955021 | | | | | | | | | | | | | | |
| chr19:2226676- | Yes | Yes | | 2349 | 1 | 97 | rs3815308 | rs2302061 | | 0.034 | 0.182 | 0.143 | 0.172 | 0.203 |
| 2226772 | | | | | | | | | | | | | | |
| chr19:30253901- | | Yes | | 768 | 2 | | rs117342492 | rs4805475 | | 0.000 | 0.221 | 0.000 | 0.104 | 0.073 |
| 30253998 | | | | | | | | | | | | | | |
| chr19:30255068- | | Yes | | 495 | 2 | 23 | rs8103966 | rs8099838 | | 0.043 | 0.310 | 0.250 | 0.232 | 0.252 |
| 30255090 | | | | | | | | | | | | | | |
| chr19:30290349- | | Yes | | 2732 | 1 | 9 | rs1473201 | rs111640872 | | 0.085 | 0.106 | 0.247 | 0.180 | 0.213 |
| 30290357 | | | | | | | | | | | | | | |
| chr19:30340381- | | Yes | | 593 | 3 | 32 | rs929813 | rs929814 | | 0.216 | 0.087 | 0.121 | 0.293 | 0.263 |
| 30340412 | | | | | | | | | | | | | | |
| chr19:30361995- | | Yes | | 290 | 2 | | rs255270 | rs255271 | | 0.184 | 0.104 | 0.037 | 0.068 | 0.012 |
| 30362112 | | | | | | | | | | | | | | |
| chr19:3119184- | Yes | Yes | | 1438 | 1 | 56 | rs308046 | rs4900 | | 0.166 | 0.233 | 0.135 | 0.101 | 0.275 |
| 3119239 | | | | | | | | | | | | | | |
| chr19:36237227- | | | Yes | 0 | 0 | 19 | rs3817622 | rs2293688 | | | | | | |
| 36237245 | | | | | | | | | | | | | | |
| chr19:41724820- | | Yes | | 2049 | 0 | 66 | rs2301236 | rs28364580 | | 0.094 | 0.179 | 0.224 | 0.148 | 0.275 |
| 41724885 | | | | | | | | | | | | | | |
| chr19:41781493- | | Yes | | 1040 | 2 | | rs8103839 | rs9304592 | | 0.067 | 0.073 | 0.000 | 0.066 | 0.064 |
| 41781579 | | | | | | | | | | | | | | |
| chr19:44352639- | | | Yes | 0 | 0 | 28 | rs1061768 | rs2356437 | rs1061769 | | | | | |
| 44352666 | | | | | | | | | | | | | | |
| chr19:4510530- | | | Yes | 0 | 0 | 31 | rs7250947 | rs7251858 | | | | | | |
| 4510560 | | | | | | | | | | | | | | |
| chr19:50919797- | Yes | Yes | | 2886 | 5 | 32 | rs3218776 | rs3218760 | | 0.125 | 0.139 | 0.075 | 0.148 | 0.275 |
| 50919828 | | | | | | | | | | | | | | |
| chr19:5210622- | Yes | | | 740 | 2 | 161 | rs2302224 | rs1143698 | | 0.166 | 0.066 | 0.126 | 0.134 | 0.090 |
| 5210782 | | | | | | | | | | | | | | |
| chr19:5210762- | Yes | | | 4185 | 0 | 21 | rs1143699 | rs1143698 | | 0.222 | 0.000 | 0.099 | 0.081 | 0.056 |
| 5210782 | | | | | | | | | | | | | | |
| chr19:5212380- | Yes | | | 1945 | 1 | 103 | rs1064300 | rs2230611 | | 0.115 | 0.000 | 0.124 | | |
| 5212482 | | | | | | | | | | | | | | |
| chr19:58131576- | | | Yes | 0 | 0 | 48 | rs10414451 | rs10413455 | | | | | | |
| 58131623 | | | | | | | | | | | | | | |
| chr19:58213952- | | | Yes | 0 | 0 | 18 | rs2074078 | rs11878316 | | | | | | |
| 58213969 | | | | | | | | | | | | | | |
| chr19:58572959- | | | Yes | 0 | 0 | 21 | rs2288274 | rs1469087 | | | | | | |
| 58572979 | | | | | | | | | | | | | | |
| chr19:7163154- | | Yes | | 810 | 2 | 77 | rs2963 | rs2245648 | | 0.186 | 0.025 | 0.065 | 0.068 | 0.141 |
| 7163230 | | | | | | | | | | | | | | |
| chr19:7166376- | Yes | Yes | | 1028 | 2 | 13 | rs2059806 | rs2229429 | | 0.179 | 0.065 | 0.191 | 0.144 | 0.262 |
| 7166388 | | | | | | | | | | | | | | |
| chr19:8148301- | | | Yes | 0 | 0 | 14 | rs17202517 | rs17160149 | | | | | | |
| 8148314 | | | | | | | | | | | | | | |
| chr19:9362297- | | | Yes | 0 | 0 | 47 | rs12980833 | rs2240927 | | | | | | |
| 9362343 | | | | | | | | | | | | | | |
| chr2:112754828- | Yes | | | 366 | 1 | 53 | rs3811632 | rs3811633 | | 0.103 | 0.106 | 0.287 | | |
| 112754880 | | | | | | | | | | | | | | |
| chr2:112754943- | Yes | | | 747 | 3 | 59 | rs3811634 | rs2230515 | | 0.104 | 0.106 | 0.287 | | |
| 112755001 | | | | | | | | | | | | | | |
| chr2:113983937- | | Yes | | 776 | 1 | 97 | rs3748915 | rs3748916 | | 0.203 | 0.086 | 0.163 | 0.135 | 0.229 |
| 113984033 | | | | | | | | | | | | | | |
| chr2:113984503- | | Yes | | 1400 | 0 | 92 | rs2241975 | rs67776659 | | 0.142 | 0.013 | 0.110 | 0.087 | 0.038 |
| 113984594 | | | | | | | | | | | | | | |
| chr2:113989236- | | Yes | | 1009 | 2 | 32 | rs2863242 | rs2863243 | | 0.017 | 0.074 | 0.163 | 0.138 | 0.183 |
| 113989267 | | | | | | | | | | | | | | |
| chr2:141259283- | Yes | | | 446 | 1 | 94 | rs35296183 | rs35164907 | | 0.021 | 0.000 | 0.048 | | |
| 141259376 | | | | | | | | | | | | | | |
| chr2:16042003- | | Yes | | 392 | 1 | 49 | rs2693006 | rs67056216 | | 0.113 | 0.177 | 0.177 | 0.159 | 0.264 |
| 16042051 | | | | | | | | | | | | | | |
| chr2:16073257- | | Yes | | 1546 | 2 | 7 | rs12986946 | rs12986949 | | 0.052 | 0.000 | 0.101 | 0.058 | 0.115 |
| 16073263 | | | | | | | | | | | | | | |
| chr2:16112814- | | Yes | | 835 | 1 | 15 | rs16863159 | rs6716344 | | 0.022 | 0.276 | 0.088 | 0.244 | 0.131 |
| 16112828 | | | | | | | | | | | | | | |
| chr2:16113594- | | Yes | | 368 | 4 | 130 | rs34339850 | rs6741005 | | 0.052 | 0.284 | 0.217 | 0.183 | 0.245 |
| 16113723 | | | | | | | | | | | | | | |
| chr2:202122956- | | Yes | | 1337 | 0 | 40 | rs3769824 | rs3769823 | | 0.000 | 0.000 | 0.047 | 0.114 | 0.043 |
| 202122995 | | | | | | | | | | | | | | |
| CHR2:231775094- | | | Yes | 0 | 0 | 51 | rs3749073 | rs1992187 | | | | | | |
| 231775144 | | | | | | | | | | | | | | |
| CHR2:239184569- | | | Yes | 0 | 0 | 13 | rs13391269 | rs10462023 | | | | | | |
| 239184581 | | | | | | | | | | | | | | |
| chr2:29416366- | Yes | | | 677 | 2 | 116 | rs1881421 | rs1881420 | | 0.240 | 0.000 | 0.150 | 0.127 | 0.027 |
| 29416481 | | | | | | | | | | | | | | |
| chr2:29416481- | Yes | | | 750 | 15 | 135 | rs1881420 | rs56132472 | | 0.078 | 0.000 | 0.123 | 0.065 | 0.024 |
| 29416615 | | | | | | | | | | | | | | |
| chr2:29446184- | Yes | Yes | | 2130 | 0 | 19 | rs2276550 | rs4622670 | | 0.259 | 0.054 | 0.236 | 0.222 | 0.203 |
| 29446202 | | | | | | | | | | | | | | |
| chr2:29446701- | | Yes | | 686 | 1 | 21 | rs12619049 | rs4665447 | | 0.412 | 0.081 | 0.026 | 0.062 | 0.015 |
| 29446721 | | | | | | | | | | | | | | |
| chr2:29447108- | | Yes | | 448 | 1 | 146 | rs4387740 | rs6723311 | | 0.390 | 0.141 | 0.254 | 0.232 | 0.173 |
| 29447253 | | | | | | | | | | | | | | |
| CHR2:33623720- | | | Yes | 0 | 0 | 15 | rs8970 | rs622716 | | | | | | |
| 33623734 | | | | | | | | | | | | | | |
| CHR2:37579937- | | | Yes | 0 | 0 | 35 | rs2302652 | rs2255991 | | | | | | |
| 37579971 | | | | | | | | | | | | | | |
| chr2:47800577- | | Yes | | 1072 | 0 | 27 | rs56239373 | rs3814360 | | 0.077 | 0.154 | 0.042 | 0.065 | 0.086 |
| 47800603 | | | | | | | | | | | | | | |
| chr2:47852559- | | Yes | | 293 | 5 | 85 | rs6722699 | rs10165802 | | 0.110 | 0.076 | 0.093 | 0.104 | 0.061 |
| 47852643 | | | | | | | | | | | | | | |
| chr2:48010488- | Yes | | | 1461 | 2 | 71 | rs1042821 | rs1042820 | | 0.020 | 0.000 | 0.175 | | |
| 48010558 | | | | | | | | | | | | | | |
| CHR2:71058184- | | | Yes | 0 | 0 | 43 | rs13421115 | rs2080390 | | | | | | |
| 71058226 | | | | | | | | | | | | | | |
| chr20:30729488- | | Yes | | 3150 | 2 | 36 | rs6089193 | rs6089194 | | 0.206 | 0.085 | 0.026 | 0.137 | 0.053 |
| 30729523 | | | | | | | | | | | | | | |
| chr20:40714307- | Yes | | | 307 | 3 | 173 | rs3092662 | rs2016647 | | 0.000 | 0.073 | 0.079 | 0.092 | 0.054 |
| 40714479 | | | | | | | | | | | | | | |
| chr20:40714479- | | Yes | | 1095 | 1 | 62 | rs2016647 | rs1569548 | | 0.114 | 0.074 | 0.242 | 0.167 | 0.138 |
| 40714540 | | | | | | | | | | | | | | |
| chr20:40714539- | Yes | | | 1134 | 12 | 2 | rs1569547 | rs1569548 | | 0.000 | 0.073 | 0.231 | | |
| 40714540 | | | | | | | | | | | | | | |
| chr20:52645534- | | | Yes | 0 | 0 | 8 | rs466264 | rs2072127 | | | | | | |
| 52645541 | | | | | | | | | | | | | | |
| chr20:57478807- | Yes | | | 711 | 8 | 133 | rs7121 | rs3730168 | | 0.186 | 0.091 | 0.286 | 0.120 | 0.169 |
| 57478939 | | | | | | | | | | | | | | |
| chr20:5904028- | | | Yes | 0 | 0 | 13 | rs742710 | rs742711 | | | | | | |
| 5904040 | | | | | | | | | | | | | | |
| chr20:62597666- | | | Yes | 0 | 0 | 29 | rs45486695 | rs817329 | | | | | | |
| 62597694 | | | | | | | | | | | | | | |
| chr20:744382- | | | Yes | 0 | 0 | 34 | rs3746803 | rs3746804 | | | | | | |
| 744415 | | | | | | | | | | | | | | |
| chr20:9543622- | Yes | Yes | | 813 | 5 | 60 | rs2297345 | rs2297346 | | 0.122 | 0.214 | 0.088 | 0.174 | 0.059 |
| 9543681 | | | | | | | | | | | | | | |
| chr21:42845374- | Yes | Yes | | 6069 | 0 | 10 | rs2298659 | rs17854725 | | 0.173 | 0.115 | 0.230 | 0.218 | 0.189 |
| 42845383 | | | | | | | | | | | | | | |
| chr21:42876400- | | Yes | | 2128 | 0 | 48 | rs7277080 | rs395584 | | 0.287 | 0.017 | 0.019 | 0.235 | 0.212 |
| 42876447 | | | | | | | | | | | | | | |
| chr21:43557698- | | | Yes | 0 | 0 | 39 | rs3819142 | rs220178 | | | | | | |
| 43557736 | | | | | | | | | | | | | | |
| chr21:46321659- | | | Yes | 0 | 0 | 19 | rs55865320 | rs5030669 | | | | | | |
| 46321677 | | | | | | | | | | | | | | |
| chr22:17589209- | | | Yes | 0 | 0 | 38 | rs879577 | rs879576 | | | | | | |
| 17589246 | | | | | | | | | | | | | | |
| chr22:17640022- | | Yes | | 1258 | 0 | 24 | rs11550530 | rs7287672 | | 0.125 | 0.035 | 0.086 | 0.130 | 0.058 |
| 17640045 | | | | | | | | | | | | | | |
| chr22:19951207- | | | Yes | 0 | 0 | 65 | rs4818 | rs4680 | | | | | | |
| 19951271 | | | | | | | | | | | | | | |
| chr22:21337266- | Yes | Yes | | 565 | 4 | 60 | rs178280 | rs13054014 | | 0.116 | 0.200 | 0.259 | 0.223 | 0.234 |
| 21337325 | | | | | | | | | | | | | | |
| chr22:21348914- | Yes | | | 1246 | 25 | 124 | rs4822790 | rs178292 | | 0.105 | 0.224 | 0.135 | 0.112 | 0.142 |
| 21349037 | | | | | | | | | | | | | | |
| chr22:21377301- | | | Yes | 0 | 0 | 34 | rs1548411 | rs1548412 | | | | | | |
| 21377334 | | | | | | | | | | | | | | |
| chr22:24158895- | Yes | Yes | | 713 | 2 | 5 | rs9608192 | rs2070457 | | 0.098 | 0.059 | 0.115 | 0.071 | 0.153 |
| 24158899 | | | | | | | | | | | | | | |
| chr22:29690246- | | Yes | | 259 | 0 | 100 | rs73156524 | rs131189 | | 0.032 | 0.281 | 0.086 | 0.053 | 0.034 |
| 29690345 | | | | | | | | | | | | | | |
| chr22:33253280- | | | Yes | 0 | 0 | 13 | rs9862 | rs11547635 | | | | | | |
| 33253292 | | | | | | | | | | | | | | |
| chr22:35817553- | | | Yes | 0 | 0 | 45 | rs2071744 | rs133431 | | | | | | |
| 35817597 | | | | | | | | | | | | | | |
| chr22:44322922- | | | Yes | 0 | 0 | 49 | rs2076213 | rs2076212 | | | | | | |
| 44322970 | | | | | | | | | | | | | | |
| chr3:122003757- | | | Yes | 0 | 0 | 13 | rs1801725 | rs1042636 | | | | | | |
| 122003769 | | | | | | | | | | | | | | |
| chr3:12649857- | | Yes | | 567 | 2 | 81 | rs2055311 | rs963959 | | 0.225 | 0.028 | 0.164 | 0.310 | 0.125 |
| 12649937 | | | | | | | | | | | | | | |
| chr3:129155451- | | | Yes | 0 | 0 | 13 | rs140693 | rs2307289 | | | | | | |
| 129155463 | | | | | | | | | | | | | | |
| chr3:136574501- | | | Yes | 0 | 0 | 21 | rs1052618 | rs1052620 | | | | | | |
| 136574521 | | | | | | | | | | | | | | |
| chr3:138327951- | | Yes | | 634 | 1 | 66 | rs61699523 | rs111398337 | | 0.167 | 0.020 | 0.028 | 0.071 | 0.110 |
| 138328016 | | | | | | | | | | | | | | |
| chr3:142277536- | | Yes | Yes | 642 | 0 | 40 | rs2227929 | rs2227930 | | 0.147 | 0.118 | 0.200 | 0.154 | 0.158 |
| 142277575 | | | | | | | | | | | | | | |
| chr3:178922222- | Yes | | | 177 | 1 | 53 | rs3729676 | rs2699896 | | 0.098 | 0.109 | 0.196 | | |
| 178922274 | | | | | | | | | | | | | | |
| chr3:178968634- | | | Yes | 1223 | 0 | 27 | rs7645550 | rs1170672 | | | | | | |
| 178968660 | | | | | | | | | | | | | | |
| chr3:178984575- | | Yes | | 2320 | 2 | 105 | rs7612684 | rs7646600 | | 0.302 | 0.011 | 0.177 | 0.131 | 0.132 |
| 178984679 | | | | | | | | | | | | | | |
| chr3:178986121- | | Yes | | 623 | 5 | 83 | rs73188921 | rs9830427 | rs9830432 | 0.158 | 0.119 | 0.054 | 0.076 | 0.190 |
| 178986203 | | | | | | | | | | | | | | |
| chr3:178990402- | | Yes | | 1179 | 1 | 61 | rs2864411 | rs6443633 | | 0.017 | 0.142 | 0.000 | 0.050 | 0.045 |
| 178990462 | | | | | | | | | | | | | | |
| chr3:183211906- | Yes | | | 536 | 2 | 121 | rs1520101 | rs2256061 | | 0.128 | 0.000 | 0.182 | | |
| 183212026 | | | | | | | | | | | | | | |
| chr3:36986932- | | Yes | | 2760 | 4 | 61 | rs2276809 | rs2276808 | | 0.073 | 0.077 | 0.115 | 0.160 | 0.216 |
| 36986992 | | | | | | | | | | | | | | |
| chr3:71247257- | | Yes | | 1098 | 0 | 48 | rs939845 | rs2037474 | | 0.163 | 0.104 | 0.064 | 0.202 | 0.044 |
| 71247304 | | | | | | | | | | | | | | |
| chr4:106196829- | Yes | Yes | | 534 | 0 | 123 | rs34402524 | rs2454206 | | 0.066 | 0.047 | 0.140 | 0.089 | 0.090 |
| 106196951 | | | | | | | | | | | | | | |
| chr4:143043340- | Yes | | | 351 | 0 | 65 | rs2270658 | rs13133767 | | 0.016 | 0.075 | 0.082 | | |
| 143043404 | | | | | | | | | | | | | | |
| chr4:143324036- | Yes | | | 209 | 2 | 59 | rs1982965 | rs1982966 | | 0.032 | 0.291 | 0.284 | 0.236 | 0.178 |
| 143324094 | | | | | | | | | | | | | | |
| chr4:156289900- | | | Yes | 0 | 0 | 18 | rs3733390 | rs3733391 | | | | | | |
| 156289917 | | | | | | | | | | | | | | |
| chr4:1745492- | | Yes | | 4202 | 2 | 9 | rs4865466 | rs4865467 | | 0.126 | 0.144 | 0.217 | 0.306 | 0.229 |
| 1745500 | | | | | | | | | | | | | | |
| chr4:1750487- | | Yes | | 1702 | 3 | 98 | rs7680647 | rs73202803 | | 0.042 | 0.161 | 0.235 | 0.180 | 0.121 |
| 1750584 | | | | | | | | | | | | | | |
| chr4:1788994- | | Yes | | 678 | 4 | 51 | rs11248077 | rs11248078 | | 0.249 | 0.233 | 0.383 | 0.346 | 0.377 |
| 1789044 | | | | | | | | | | | | | | |
| chr4:1796629- | | Yes | | 319 | 1 | 8 | rs3135841 | rs3135842 | | 0.254 | 0.051 | 0.094 | 0.141 | 0.061 |
| 1796636 | | | | | | | | | | | | | | |
| chr4:1797741- | | Yes | | 995 | 4 | 112 | rs3135848 | rs743682 | | 0.227 | 0.056 | 0.092 | 0.144 | 0.062 |
| 1797852 | | | | | | | | | | | | | | |
| chr4:187534362- | Yes | Yes | | 2353 | 0 | 14 | rs2249916 | rs2249917 | | 0.195 | 0.281 | 0.110 | 0.189 | 0.084 |
| 187534375 | | | | | | | | | | | | | | |
| chr4:187629497- | Yes | Yes | | 1727 | 0 | 42 | rs458021 | rs3733413 | | 0.128 | 0.085 | 0.070 | 0.091 | 0.031 |
| 187629538 | | | | | | | | | | | | | | |
| chr4:54269096- | | Yes | | 557 | 1 | 78 | rs10001201 | rs62325166 | | 0.050 | 0.133 | 0.140 | 0.105 | 0.046 |
| 54269173 | | | | | | | | | | | | | | |
| chr4:54657737- | | Yes | | 288 | 5 | | rs28489910 | rs4864823 | | 0.233 | 0.111 | 0.209 | 0.226 | 0.148 |
| 54657790 | | | | | | | | | | | | | | |
| chr4:55208737- | | Yes | | 284 | 3 | 52 | rs2412560 | rs10018115 | rs73234206 | 0.202 | 0.247 | 0.200 | 0.270 | 0.317 |
| 55208788 | | | | | | | | | | | | | | |
| chr4:55501109- | | Yes | | 357 | 5 | 87 | rs6554196 | rs6554197 | | 0.110 | 0.110 | 0.200 | 0.163 | 0.223 |
| 55501195 | | | | | | | | | | | | | | |
| chr4:55582037- | | Yes | | 714 | 3 | | rs76272262 | rs3134889 | | 0.040 | 0.172 | 0.036 | 0.051 | 0.081 |
| 55582068 | | | | | | | | | | | | | | |
| chr4:55619846- | | Yes | | 892 | 3 | 14 | rs11732442 | rs4353958 | | 0.125 | 0.109 | 0.109 | 0.069 | 0.212 |
| 55619859 | | | | | | | | | | | | | | |
| chr4:55982752- | | Yes | | 651 | 1 | 33 | rs11133360 | rs34945396 | | 0.044 | 0.204 | 0.194 | 0.144 | 0.190 |
| 55982784 | | | | | | | | | | | | | | |
| chr4:56026865- | | Yes | | 565 | 1 | 50 | rs4864958 | rs75371420 | rs34743464 | 0.216 | 0.200 | 0.284 | 0.180 | 0.453 |
| 56026914 | | | | | | | | | | | | | | |
| chr5:147024476- | | | Yes | 0 | 0 | 34 | rs2116766 | rs2116765 | | ND | ND | ND | ND | ND |
| 147024509 | | | | | | | | | | | | | | |
| chr5:148206440- | | | Yes | 0 | 0 | 34 | rs1042713 | rs1042714 | | | | | | |
| 148206473 | | | | | | | | | | | | | | |
| chr5:149456772- | Yes | Yes | | 1109 | 3 | 40 | rs60844779 | rs3829987 | | 0.223 | 0.068 | 0.031 | 0.215 | 0.051 |
| 149456811 | | | | | | | | | | | | | | |
| chr5:149495287- | Yes | | | 1074 | 3 | 109 | rs2229561 | rs246388 | | ND | ND | ND | ND | ND |
| 149495395 | | | | | | | | | | | | | | |
| chr5:150666933- | | | Yes | 0 | 0 | 30 | rs375396 | rs12520516 | | | | | | |
| 150666962 | | | | | | | | | | | | | | |
| chr5:150901613- | | | Yes | 0 | 0 | 18 | rs2053028 | rs3734049 | | | | | | |
| 150901630 | | | | | | | | | | | | | | |
| chr5:174870150- | | | Yes | 0 | 0 | 47 | rs4532 | rs5326 | | | | | | |
| 174870196 | | | | | | | | | | | | | | |
| chr5:176517326- | Yes | | | 652 | 3 | 136 | rs422421 | rs446382 | | 0.169 | 0.000 | 0.078 | 0.040 | 0.033 |
| 176517461 | | | | | | | | | | | | | | |
| chr5:176523562- | Yes | Yes | | 1990 | 0 | 36 | rs31777 | rs31776 | | 0.137 | 0.000 | 0.076 | 0.038 | 0.033 |
| 176523597 | | | | | | | | | | | | | | |
| chr5:176531772- | | Yes | | 284 | 3 | 86 | rs7708357 | rs165943 | | 0.168 | 0.046 | 0.242 | 0.248 | 0.183 |
| 176531857 | | | | | | | | | | | | | | |
| chrs:176721198- | Yes | | | 1806 | 1 | 75 | rs28580074 | rs11740250 | | 0.011 | 0.000 | 0.119 | | |
| 176721272 | | | | | | | | | | | | | | |
| chrs:180046209- | Yes | | | 765 | 12 | 136 | rs446003 | rs448012 | | 0.100 | 0.057 | 0.083 | 0.075 | 0.135 |
| 180046344 | | | | | | | | | | | | | | |
| chr5:180051003- | Yes | | | 2483 | 2 | 116 | rs307826 | rs728986 | | 0.015 | 0.000 | 0.037 | | |
| 180051118 | | | | | | | | | | | | | | |
| chr5:180057231- | Yes | | | 1518 | 0 | 63 | rs3736061 | rs34221241 | | 0.000 | 0.000 | 0.081 | | |
| 180057293 | | | | | | | | | | | | | | |
| chr5:231111- | Yes | Yes | | 2366 | 1 | 33 | rs1126417 | rs2288459 | | 0.164 | 0.058 | 0.111 | 0.241 | 0.079 |
| 231143 | | | | | | | | | | | | | | |
| chr5:35861068- | Yes | Yes | | 351 | 3 | 92 | rs1494558 | rs11567705 | rs969128 | 0.328 | 0.191 | 0.413 | 0.349 | 0.239 |
| 35861159 | | | | | | | | | | | | | | |
| chr5:35871190- | Yes | Yes | | 255 | 1 | 84 | rs1494555 | rs2228141 | | 0.069 | 0.153 | 0.144 | 0.166 | 0.062 |
| 35871273 | | | | | | | | | | | | | | |
| chr5:56178111- | | Yes | | 473 | 0 | | rs3822625 | rs832583 | | 0.119 | 0.108 | 0.075 | 0.078 | 0.055 |
| 56178217 | | | | | | | | | | | | | | |
| chr5:57754808- | Yes | | | 359 | 2 | 44 | rs697133 | rs702722 | | 0.230 | 0.105 | 0.104 | 0.069 | 0.098 |
| 57754851 | | | | | | | | | | | | | | |
| chr5:67477132- | | Yes | | 371 | 0 | | rs34721946 | rs34166422 | rs73126524 | 0.017 | 0.247 | 0.035 | 0.105 | 0.072 |
| 67477234 | | | | | | | | | | | | | | |
| chr5:67492589- | | Yes | | 677 | 2 | 64 | rs13188623 | rs58409263 | | 0.105 | 0.293 | 0.121 | 0.180 | 0.118 |
| 67492652 | | | | | | | | | | | | | | |
| chr5:67517563- | | Yes | | 275 | 1 | 84 | rs6449959 | rs831227 | | 0.243 | 0.018 | 0.187 | 0.161 | 0.100 |
| 67517646 | | | | | | | | | | | | | | |
| chr5:67522722- | Yes | Yes | | 262 | 1 | 130 | rs706713 | rs706714 | | 0.130 | 0.051 | 0.012 | 0.029 | 0.060 |
| 67522851 | | | | | | | | | | | | | | |
| chr5:67534039- | | Yes | | 887 | 0 | 19 | rs7709243 | rs10940158 | rs12652661 | 0.216 | 0.154 | 0.212 | 0.272 | 0.097 |
| 67534057 | | | | | | | | | | | | | | |
| chr5:67553771- | | Yes | | 584 | 1 | 57 | rs6893676 | rs34303 | | 0.090 | 0.168 | 0.173 | 0.143 | 0.106 |
| 67553827 | | | | | | | | | | | | | | |
| chr6:117725448- | Yes | | | 277 | 4 | 131 | rs1998206 | rs2243378 | | 0.076 | 0.181 | 0.150 | 0.143 | 0.197 |
| 117725578 | | | | | | | | | | | | | | |
| chr6:117730673- | Yes | | | 158 | 0 | 147 | rs17634067 | rs2273601 | | 0.040 | 0.000 | 0.111 | 0.052 | 0.096 |
| 117730819 | | | | | | | | | | | | | | |
| chr6:152382311- | Yes | | | 279 | 2 | 15 | rs2273206 | rs2273207 | | 0.137 | 0.039 | 0.026 | 0.039 | 0.055 |
| 152382325 | | | | | | | | | | | | | | |
| chr6:167754702- | | | Yes | 0 | 0 | 20 | rs909546 | rs9457304 | | | | | | |
| 167754721 | | | | | | | | | | | | | | |
| chr6:26056549- | Yes | Yes | | 524 | 2 | 160 | rs10425 | rs2230653 | rs12204800 | 0.048 | 0.309 | 0.227 | 0.344 | 0.256 |
| 26056708 | | | | | | | | | | | | | | |
| chr6:29913201- | | | Yes | 0 | 0 | 66 | rs41557912 | rs1061156 | | | | | | |
| 29913266 | | | | | | | | | | | | | | |
| chr6:30080231- | | | Yes | 0 | 0 | 44 | rs3734838 | rs2517598 | | | | | | |
| 30080274 | | | | | | | | | | | | | | |
| chr6:30865115- | Yes | Yes | | 461 | 5 | 90 | rs2239517 | rs2267641 | | 0.120 | 0.244 | 0.038 | 0.063 | 0.094 |
| 30865204 | | | | | | | | | | | | | | |
| chr6:30993533- | | | Yes | 0 | 0 | 58 | rs2523898 | rs4713420 | rs12179536 | | | | | |
| 30993590 | | | | | | | | | | | | | | |
| chr6:31170514- | | | Yes | 0 | 0 | 15 | rs9263870 | rs9263871 | | | | | | |
| 31170528 | | | | | | | | | | | | | | |
| chr6:31930441- | | | Yes | 0 | 0 | 22 | rs592229 | rs429608 | | | | | | |
| 31930462 | | | | | | | | | | | | | | |
| chr6:32188603- | Yes | Yes | | 1185 | 1 | 40 | rs520803 | rs520692 | rs520688 | 0.000 | 0.047 | 0.000 | 0.000 | 0.011 |
| 32188642 | | | | | | | | | | | | | | |
| chr6:32190390- | | Yes | | 2363 | 5 | 95 | rs915894 | rs8192569 | | 0.330 | 0.232 | 0.102 | 0.141 | 0.205 |
| 32190484 | | | | | | | | | | | | | | |
| chr6:33141253- | | | Yes | 0 | 0 | 28 | rs9277932 | rs2855430 | | | | | | |
| 33141280 | | | | | | | | | | | | | | |
| chr6:36291985- | | | Yes | 0 | 0 | 23 | rs7751919 | rs7751928 | | | | | | |
| 36292007 | | | | | | | | | | | | | | |
| chr6:4069133- | | | Yes | 0 | 0 | 34 | rs10485172 | rs595413 | | ND | ND | ND | ND | ND |
| 4069166 | | | | | | | | | | | | | | |
| chr6:41924853- | | Yes | | 922 | 2 | 79 | rs4623235 | rs16895130 | | 0.095 | 0.110 | 0.210 | 0.156 | 0.138 |
| 41924931 | | | | | | | | | | | | | | |
| chr6:42013020- | | Yes | | 530 | 0 | | rs9381126 | rs6919122 | rs6942118 | 0.351 | 0.421 | 0.381 | 0.504 | 0.390 |
| 42013049 | | | | | | | | | | | | | | |
| chr6:42039487- | | Yes | | 651 | 3 | 56 | rs9349215 | rs66472208 | | 0.023 | 0.245 | 0.020 | 0.048 | 0.127 |
| 42039542 | | | | | | | | | | | | | | |
| chr6:42039551- | | Yes | | 292 | 1 | 116 | rs66489927 | rs7763360 | rs2492927 | 0.192 | 0.148 | 0.300 | 0.248 | 0.322 |
| 42039666 | | | | | | | | | | | | | | |
| chr6:42052577- | | Yes | | 305 | 0 | 91 | rs9357387 | rs2493841 | rs9381136 | 0.050 | 0.163 | 0.176 | 0.161 | 0.139 |
| 42052667 | | | | | | | | | | | | | | |
| chr7:100410597- | Yes | | | 1469 | 8 | 61 | rs2230585 | rs770657085 | | 0.164 | 0.056 | 0.000 | 0.043 | 0.156 |
| 100410657 | | | | | | | | | | | | | | |
| chr7:100416139- | | Yes | | 1438 | 3 | | rs3857809 | rs144173 | | 0.185 | 0.059 | 0.000 | 0.301 | 0.173 |
| 100416250 | | | | | | | | | | | | | | |
| chr7:100677455- | | | Yes | 0 | 0 | 69 | rs61075804 | rs10238201 | | | | | | |
| 100677523 | | | | | | | | | | | | | | |
| chr7:116336880- | | Yes | | 666 | 1 | 68 | rs2237708 | rs39749 | | 0.036 | 0.209 | 0.257 | 0.228 | 0.242 |
| 116336947 | | | | | | | | | | | | | | |
| chr7:116471122- | | Yes | | 297 | 4 | 106 | rs41773 | rs62470772 | | 0.129 | 0.093 | 0.206 | 0.115 | 0.148 |
| 116471227 | | | | | | | | | | | | | | |
| chr7:21640361- | | | Yes | 0 | 0 | 45 | rs10269582 | rs10224537 | | | | | | |
| 21640405 | | | | | | | | | | | | | | |
| chr7:27196069- | | | Yes | 0 | 0 | 45 | rs2301720 | rs2301721 | | | | | | |
| 27196113 | | | | | | | | | | | | | | |
| chr7:30795288- | | | Yes | 0 | 0 | 44 | rs2302339 | rs2302340 | | | | | | |
| 30795331 | | | | | | | | | | | | | | |
| chr7:4213975- | | | Yes | 0 | 0 | 49 | rs671694 | rs886731 | | | | | | |
| 4214023 | | | | | | | | | | | | | | |
| chr7:55220177- | | Yes | Yes | 1118 | 0 | 26 | rs11506105 | rs845561 | | 0.115 | 0.265 | 0.254 | 0.304 | 0.413 |
| 55220202 | | | | | | | | | | | | | | |
| chr7:55251541- | | Yes | | 672 | 4 | 108 | rs2877261 | rs13222385 | rs11771471 | 0.200 | 0.076 | 0.233 | 0.183 | 0.090 |
| 55251648 | | | | | | | | | | | | | | |
| chr7:6026775- | Yes | | | 720 | 19 | 168 | rs2228006 | rs1805323 | | 0.000 | 0.122 | 0.046 | 0.017 | 0.106 |
| 6026942 | | | | | | | | | | | | | | |
| chr7:6026942- | | Yes | | 3560 | 3 | 47 | rs1805323 | rs1805321 | | 0.000 | 0.303 | 0.046 | 0.017 | 0.153 |
| 6026988 | | | | | | | | | | | | | | |
| chr7:78119109- | Yes | | | 330 | 2 | 91 | rs3735442 | rs1990577 | | ND | ND | ND | ND | ND |
| 78119199 | | | | | | | | | | | | | | |
| chr8:128700175- | | Yes | | 496 | 2 | 59 | rs13282849 | rs7005394 | | 0.208 | 0.179 | 0.063 | 0.084 | 0.201 |
| 128700233 | | | | | | | | | | | | | | |
| chr8:128713221- | | Yes | | 796 | 5 | 144 | rs28548827 | rs7820045 | | 0.254 | 0.057 | 0.028 | 0.101 | 0.111 |
| 128713364 | | | | | | | | | | | | | | |
| chr8:128889285- | | Yes | | 1835 | 1 | | rs6470587 | rs6470588 | | 0.081 | 0.165 | 0.210 | 0.202 | 0.230 |
| 128889371 | | | | | | | | | | | | | | |
| CHR8:142490120- | | | Yes | 0 | 0 | 47 | rs2748416 | rs7838192 | | | | | | |
| 142490166 | | | | | | | | | | | | | | |
| CHR8:145639681- | | | Yes | 0 | 0 | 46 | rs1871534 | rs2272662 | | | | | | |
| 145639726 | | | | | | | | | | | | | | |
| chr8:145737636- | | Yes | | 485 | 0 | | rs4925828 | rs4251691 | | 0.000 | 0.203 | 0.000 | 0.072 | 0.000 |
| 145737816 | | | | | | | | | | | | | | |
| chr8:30999122- | Yes | Yes | | 554 | 3 | 2 | rs3024239 | rs2737335 | | 0.149 | 0.024 | 0.060 | 0.032 | 0.085 |
| 30999123 | | | | | | | | | | | | | | |
| chr8:31024638- | Yes | Yes | | 432 | 0 | 17 | rs1801196 | rs1346044 | | 0.147 | 0.104 | 0.266 | 0.173 | 0.283 |
| 31024654 | | | | | | | | | | | | | | |
| chr8:38299624- | | Yes | | 1668 | 5 | 92 | rs60527016 | rs6987534 | | 0.028 | 0.286 | 0.236 | 0.219 | 0.076 |
| 38299715 | | | | | | | | | | | | | | |
| chr8:38310910- | | Yes | | 1289 | 0 | 92 | rs10958700 | rs4733930 | | 0.029 | 0.323 | 0.260 | 0.249 | 0.074 |
| 38311001 | | | | | | | | | | | | | | |
| chr8:38350292- | | Yes | | 580 | 2 | 24 | rs35305468 | rs7830964 | | 0.039 | 0.249 | 0.180 | 0.118 | 0.138 |
| 38350315 | | | | | | | | | | | | | | |
| chr8:38361379- | | Yes | | 1456 | 2 | 52 | rs328294 | rs328293 | | 0.309 | 0.172 | 0.126 | 0.115 | 0.283 |
| 38361430 | | | | | | | | | | | | | | |
| chr8:90958422- | Yes | | | 182 | 1 | 109 | rs1061302 | rs2308962 | | 0.097 | 0.000 | 0.000 | 0.000 | 0.000 |
| 90958530 | | | | | | | | | | | | | | |
| chr9:117166206- | | | Yes | 0 | 0 | 41 | rs2274158 | rs2274159 | | | | | | |
| 117166246 | | | | | | | | | | | | | | |
| chr9:125315542- | | | Yes | 0 | 0 | 16 | rs1831369 | rs1831370 | | | | | | |
| 125315557 | | | | | | | | | | | | | | |
| chr9:134385435- | | | Yes | 0 | 0 | 2 | rs3887873 | rs2296949 | | | | | | |
| 134385436 | | | | | | | | | | | | | | |
| chr9:136412255- | | | Yes | 0 | 0 | 42 | rs2073876 | rs2073877 | | | | | | |
| 136412296 | | | | | | | | | | | | | | |
| chr9:139401504- | | Yes | | 1346 | 1 | 74 | rs3124596 | rs7870145 | rs3829116 | 0.310 | 0.000 | 0.163 | 0.117 | 0.264 |
| 139401577 | | | | | | | | | | | | | | |
| chr9:139403268- | | Yes | | 500 | 1 | 13 | rs3125000 | rs11145765 | | 0.046 | 0.000 | 0.095 | | |
| 139403280 | | | | | | | | | | | | | | |
| chr9:139405093- | Yes | Yes | | 626 | 3 | 169 | rs36119806 | rs3125001 | | 0.150 | 0.012 | 0.102 | 0.065 | 0.184 |
| 139405261 | | | | | | | | | | | | | | |
| chr9:139410424- | Yes | Yes | | 327 | 2 | 166 | rs3125006 | rs4880099 | | 0.088 | 0.052 | 0.115 | 0.068 | 0.215 |
| 139410589 | | | | | | | | | | | | | | |
| chr9:139411714- | Yes | | | 428 | 5 | 167 | rs11145767 | rs9411254 | | 0.209 | 0.000 | 0.000 | 0.025 | 0.000 |
| 139411880 | | | | | | | | | | | | | | |
| chr9:21968159- | Yes | | | 213 | 0 | 41 | rs3088440 | rs11515 | | 0.164 | 0.019 | 0.079 | 0.078 | 0.052 |
| 21968199 | | | | | | | | | | | | | | |
| chr9:5408242- | | Yes | | 344 | 3 | 117 | rs10758685 | rs10975098 | rs10975099 | 0.084 | 0.349 | 0.257 | 0.320 | 0.409 |
| 5408358 | | | | | | | | | | | | | | |
| chr9:5415025- | | Yes | | 372 | 3 | | rs78298180 | rs10758687 | | 0.104 | 0.161 | 0.054 | 0.052 | 0.199 |
| 5415111 | | | | | | | | | | | | | | |
| chr9:5420254- | | Yes | | 1180 | 1 | 13 | rs10121219 | rs11790878 | | 0.064 | 0.227 | 0.222 | 0.248 | 0.218 |
| 5420266 | | | | | | | | | | | | | | |
| chr9:5458035- | | Yes | | 323 | 3 | 61 | rs7042084 | rs10481593 | | 0.268 | 0.132 | 0.220 | 0.249 | 0.131 |
| 5458095 | | | | | | | | | | | | | | |
| chr9:5484100- | | Yes | | 395 | 4 | 104 | rs11793113 | rs11790610 | rs10122509 | 0.139 | 0.151 | 0.094 | 0.084 | 0.167 |
| 5484203 | | | | | | | | | | | | | | |
| chr9:87478135- | | Yes | | 1016 | 4 | 38 | rs7048015 | rs10780690 | | 0.023 | 0.251 | 0.184 | 0.258 | 0.216 |
| 87478172 | | | | | | | | | | | | | | |
| chr9:93639846- | Yes | | | 487 | 6 | 128 | rs290223 | rs2290888 | | ND | ND | ND | ND | ND |
| 93639973 | | | | | | | | | | | | | | |
| chr9:93641175- | Yes | | | 693 | 2 | 25 | rs2306041 | rs2306040 | | 0.062 | 0.000 | 0.064 | | |
| 93641199 | | | | | | | | | | | | | | |
| chr9:98238358- | Yes | Yes | | 3840 | 0 | 22 | rs2066836 | rs1805155 | | 0.011 | 0.083 | 0.109 | 0.076 | 0.060 |
| 98238379 | | | | | | | | | | | | | | |
| chrX:23019317- | | | Yes | 0 | 0 | 30 | rs5925720 | rs5926203 | | | | | | |
| 23019346 | | | | | | | | | | | | | | |

[symbol]

indicates data missing or illegible when filed

TABLE 8
Observed 3rd MH Frequency (x2).

| Observed 3rd MH Frequency (x2) | | | | | | | | | |
|---|---|---|---|---|---|---|---|---|---|
| | 1 | 1.5 | 2 | 2.5 | 3 | 4 | 5 | 7 | 9 |

Asian

| | | | | | | | | | | |
|---|---|---|---|---|---|---|---|---|---|---|
| In | 0.5 | 8 | 0 | 0 | 0 | 0 | 0 | 0 | 0 | 0 |
| silico | 1 | 15 | 2 | 0 | 0 | 0 | 0 | 0 | 0 | 0 |
| Mixing | 1.5 | 15 | 12 | 0 | 0 | 0 | 0 | 0 | 0 | 0 |
| Levels | 2 | 15 | 14 | 10 | 0 | 0 | 0 | 0 | 0 | 0 |
| | 2.5 | 15 | 15 | 15 | 8 | 0 | 0 | 0 | 0 | 0 |
| | 3 | 15 | 15 | 15 | 15 | 6 | 0 | 0 | 0 | 0 |
| | 4 | 15 | 15 | 15 | 15 | 15 | 3 | 0 | 0 | 0 |
| | 5 | 15 | 15 | 15 | 15 | 15 | 15 | 1 | 0 | 0 |
| | 10 | 15 | 15 | 15 | 15 | 15 | 15 | 15 | 15 | 9 |

African

| | | | | | | | | | | |
|---|---|---|---|---|---|---|---|---|---|---|
| In | 0.5 | 3 | 0 | 0 | 0 | 0 | 0 | 0 | 0 | 0 |
| silico | 1 | 15 | 0 | 0 | 0 | 0 | 0 | 0 | 0 | 0 |
| Mixing | 1.5 | 15 | 10 | 0 | 0 | 0 | 0 | 0 | 0 | 0 |
| Levels | 2 | 15 | 14 | 5 | 0 | 0 | 0 | 0 | 0 | 0 |
| | 2.5 | 15 | 15 | 15 | 4 | 0 | 0 | 0 | 0 | 0 |
| | 3 | 15 | 15 | 15 | 14 | 5 | 0 | 0 | 0 | 0 |
| | 4 | 15 | 15 | 15 | 15 | 13 | 1 | 0 | 0 | 0 |
| | 5 | 15 | 15 | 15 | 15 | 15 | 12 | 2 | 0 | 0 |
| | 10 | 15 | 15 | 15 | 15 | 15 | 15 | 15 | 14 | 7 |

European

| | | | | | | | | | | |
|---|---|---|---|---|---|---|---|---|---|---|
| | 0.5 | 8 | 0 | 0 | 0 | 0 | 0 | 0 | 0 | 0 |
| In | 1 | 15 | 4 | 0 | 0 | 0 | 0 | 0 | 0 | 0 |
| silico | 1.5 | 15 | 13 | 4 | 0 | 0 | 0 | 0 | 0 | 0 |
| Mixing | 2 | 15 | 15 | 12 | 0 | 0 | 0 | 0 | 0 | 0 |
| Levels | 2.5 | 15 | 15 | 15 | 8 | 0 | 0 | 0 | 0 | 0 |
| | 3 | 15 | 15 | 15 | 13 | 4 | 0 | 0 | 0 | 0 |
| | 4 | 15 | 15 | 15 | 14 | 14 | 3 | 0 | 0 | 0 |
| | 5 | 15 | 15 | 15 | 15 | 15 | 12 | 1 | 0 | 0 |
| | 10 | 15 | 15 | 15 | 15 | 15 | 15 | 15 | 13 | 7 |

Mixed

| | | | | | | | | | | |
|---|---|---|---|---|---|---|---|---|---|---|
| In | 0.5 | 5 | 0 | 0 | 0 | 0 | 0 | 0 | 0 | 0 |
| silico | 1 | 15 | 3 | 0 | 0 | 0 | 0 | 0 | 0 | 0 |
| Mixing | 1.5 | 15 | 14 | 0 | 0 | 0 | 0 | 0 | 0 | 0 |
| Levels | 2 | 15 | 15 | 11 | 0 | 0 | 0 | 0 | 0 | 0 |
| | 2.5 | 15 | 15 | 15 | 7 | 1 | 0 | 0 | 0 | 0 |
| | 3 | 15 | 15 | 15 | 15 | 6 | 0 | 0 | 0 | 0 |
| | 4 | 15 | 15 | 15 | 15 | 15 | 2 | 0 | 0 | 0 |
| | 5 | 15 | 15 | 15 | 15 | 15 | 14 | 0 | 0 | 0 |
| | 10 | 15 | 15 | 15 | 15 | 15 | 15 | 15 | 14 | 9 |

All (%)

| | | | | | | | | | | |
|---|---|---|---|---|---|---|---|---|---|---|
| In | 0.5 | 40 | 0 | 0 | 0 | 0 | 0 | 0 | 0 | 0 |
| silico | 1 | 100 | 15 | 0 | 0 | 0 | 0 | 0 | 0 | 0 |
| Mixing | 1.5 | 100 | 82 | 7 | 0 | 0 | 0 | 0 | 0 | 0 |
| Levels | 2 | 100 | 97 | 63 | 0 | 0 | 0 | 0 | 0 | 0 |
| | 2.5 | 100 | 100 | 100 | 45 | 2 | 0 | 0 | 0 | 0 |
| | 3 | 100 | 100 | 100 | 95 | 35 | 0 | 0 | 0 | 0 |
| | 4 | 100 | 100 | 100 | 98 | 95 | 15 | 0 | 0 | 0 |
| | 5 | 100 | 100 | 100 | 100 | 100 | 88 | 7 | 0 | 0 |
| | 10 | 100 | 100 | 100 | 100 | 100 | 100 | 100 | 93 | 53 |

Although the invention has been described with reference to the above examples, it will be understood that modifications and variations are encompassed within the spirit and scope of the invention. Accordingly, the invention is limited only by the following claims.

1. A method of identifying microhaplotypes in a genome comprising:
a) identifying a region of interest of the genome;
b) detecting single base pair substitutions (SBSs) within the region of interest thereby generating multiple sequence variant sets;
c) analyzing each variant set for linkage disequilibrium to identify candidate microhaplotypes; and
d) identifying candidate microhaplotypes.

2. The method of claim 1, further comprising detecting SBSs in regions flanking the region of interest.

3. The method of claim 2, wherein the regions flanking the region of interest comprise less than about 50, 100, 150, 180 or 200 nucleotide base pairs capable of being sequenced by a short read sequencer.

4. The method of claim 2, wherein the regions flanking the region of interest comprise less than about 10,000 nucleotide base pairs capable of being sequenced by a long read sequencer.

5. The method of claim 1, wherein the region of interest of a) has SBSs at a frequency of between about 10-90%.

6. The method of claim 2, wherein the regions flanking the region of interest have SBSs at a frequency of between about 5-95%.

7. The method of claim 1, further comprising calibrating cutoff values for candidate microhaplotypes for assessing contamination of a sample.

8. The method of claim 6, wherein only DNA sequence reads overlapping the candidate microhaplotypes are used for calculating thresholds for contamination detection and degree of contamination.

9. The method of claim 8, wherein the DNA sequences being used to calibrate thresholds for contamination detection and degree of contamination are mixed pairwise in silico, alternately using each DNA sequence as primary sample and contaminant.

10. The method of claim 8, wherein the number and genotype of SNP sets with 1 and/or 2 microhaplotypes are compared between different individuals to assess identity or contamination.

11. The method of claim 7, further comprising assessing sample contamination utilizing determined cutoff values for frequency of candidate microhaplotypes having single nucleotide polymorphism (SNP) sets with at least 3 microhaplotypes.

12. The method of claim 11, further comprising assessing sample contamination utilizing determined cutoff values for frequency of candidate microhaplotypes having SNP sets with at least 4 or more microhaplotypes.

13. The method of claim 1, wherein the candidate microhaplotypes correspond to one or more genomic regions selected from those set forth in Tables 5, 6, or 7.

14. The method of claim 7, wherein the sample comprises DNA from a tumor or a liquid biopsy.

15. The method of claim 7, wherein the sample comprises DNA extracted from a formalin fixed paraffin embedded block, slide, or curl.

16. The method of claim 14, wherein the liquid biopsy is from amniotic fluid, aqueous humour, vitreous humour, blood, whole blood, fractionated blood, plasma, serum, breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, prostatic fluid, nipple aspirate fluid, lachrymal fluid, perspiration, cheek swabs, cell lysate, gastrointestinal fluid, biopsy tissue and urine or other biological fluid.

17. The method of claim 14, wherein the sample is from a circulating tumor cell.

18. The method of claim 7, wherein calibrating comprises analysis of the candidate microhaplotype in multiple samples obtained from humans of different ethnicities.

19. The method of claim 1, wherein the candidate microhaplotypes comprise SNP sets having at least 3, 4 or more sets of SNP sequence variants.

20. The method of claim 1, wherein the region of interest is within a gene, an intron and/or an exon or between genes.

21. The method of claim 1, wherein the region of interest is within an exome.

22. The method of claim 1, further comprising isolating the DNA comprising the candidate microhaplotypes.

23. The method of claim 1, wherein the genome is from a human.

24. The method of claim 1, further comprising assessing sample contamination by analyzing median, average or other measure of microhaplotype frequency of haplotypes within SNP sets with at least 3 or 4 microhaplotypes.

25-31. (canceled)

32. Use of the method of claim 1 to assess quality of samples from a particular source or vendor or technician preparing or sequencing samples.

33. A method for detecting single nucleotide polymorphism (SNP) sets having at least three microhaplotypes from multiple subjects present in a sample comprising:
a) identifying microhaplotypes in a genome in the sample, wherein identifying comprises:
i) identifying a region of interest of the genome;
ii) detecting single base pair substitutions (SBSs) within the region of interest thereby generating multiple sequence variant sets; and
iii) analyzing each variant set for linkage disequilibrium to identify microhaplotypes;
b) determining the number of SNP sets having at least 3 microhaplotypes in the sample; and
c) quantitating the frequency of the SNP sets with greater than 2 microhaplotypes to determine the presence of DNA from multiple subjects in the sample, thereby detecting DNA from multiple subjects in the sample.

34. The method of claim 33, further comprising isolating DNA comprising the microhaplotypes from the sample.

35. The method of claim 33, further comprising detecting SBSs in regions of the genome flanking the region of interest.

36. The method of claim 35, wherein the regions flanking the region of interest comprises less than about 50, 100, 150, 180 or 200 nucleotide base pairs capable of being sequenced by a short read sequencer.

37. The method of claim 35, wherein the regions flanking the region of interest comprises less than about 10,000 nucleotide base pairs capable of being sequenced by a long read sequencer.

38-48. (canceled)

49. A method for detecting single nucleotide polymorphism (SNP) sets having at least three microhaplotypes from multiple subjects present in a sample comprising:
a) determining the presence or absence of SNP sets having more than two microhaplotypes in the sample, wherein the SNP sets comprise multiple single base pair substitutions and correspond to a genomic region selected from regions set forth in Tables 5 and 6 and 7; and
b) quantitating the frequency of the SNP sets to determine the presence of DNA from multiple subjects in the sample, thereby detecting SNP sets having at least 3 microhaplotypes from multiple subjects in the sample.

50-90. (canceled)
